(19) United States
(12) Patent Application Publication
(10) Pub. No.: US 2025/0099120 A1
(43) Pub. Date: Mar. 27, 2025

(54) ENDOVASCULAR MEDICAL DEVICES AND ASSOCIATED DEVICES, SYSTEMS, AND METHODS

(71) Applicant: Intervene, Inc., South San Francisco, CA (US)

(72) Inventors: Daniel T. Lagoe, Pacifica, CA (US); Jeffrey M. Elkins, Woodside, CA (US); Kent D. Dell, Lincoln, CA (US); Eric Dooley, Halfmoon Bay, CA (US); Herbert M. Mendoza, South San Francisco, CA (US); Abha Chinubhai, Fremont, CA (US); Abraham Van Scoit, Los Gatos, CA (US)

(21) Appl. No.: 18/892,536

(22) Filed: Sep. 22, 2024

Related U.S. Application Data
(60) Provisional application No. 63/584,441, filed on Sep. 21, 2023.
(60) Provisional application No. 63/622,799, filed on Jan. 19, 2024.

(51) Int. Cl. A61B 17/221

(52) U.S. Cl. CPC A61B 17/221; A61B 2017/2212

(57) ABSTRACT

Endovascular treatment devices are disclosed herein. According to some embodiments, the present technology includes an elongate control member and an expandable basket coupled to a distal portion of the control member. The basket can comprise a plurality of braided filaments having a proximal opening surrounding by a proximal edge. The basket can further comprise first and second legs having respective proximal ends coupled to the control member at a connection and distal ends at the proximal edge of the basket. Each of the first and second legs can be formed of bundled portions of the filaments. The first and second legs can be configured to position the basket within the blood vessel lumen independent of the path of the control member such that the proximal edge of the basket remains in contact with an inner surface of the blood vessel lumen when the basket is positioned around a curve.

### CROSS-REFERENCE TO RELATED APPLICATION(S)

This application claims the benefit of priority to U.S. Provisional Application No. 63/584,441 filed Sep. 21, 2023, titled ENDOVASCULAR EMBOLIC COLLECTION AND REMOVAL BASKET, and U.S. Provisional Application No. 63/622,799, filed Jan. 19, 2024, titled ENDOVASCULAR EMBOLIC COLLECTION AND REMOVAL BASKET, both of which are disclosed by reference herein in their entireties.

### TECHNICAL FIELD

The present technology relates generally to endovascular treatment systems and associated devices and methods.

### BACKGROUND

Deep vein thrombosis (DVT) is a condition comprising a blood clot in a deep vein, usually a leg vein though they can also occur in arm veins. Symptoms include pain, swelling, tenderness, and/or discoloration in the affected limb. If untreated, it can lead to worsening of symptoms and complications such as post-thrombotic syndrome with symptoms of chronic pain, swelling, and skin discoloration, or pulmonary embolism (PE), a very serious and life-threatening condition. Pharmacologic treatments include blood-thinning medications or thrombolytic drugs. More recently, percutaneous catheters have been developed for the more rapid removal of clot to remove the blockage and prevent PE. These include catheters which can deliver thrombolytic agents to the site of the clot, in some cases in combination with aspiration and/or the disruption of the clot into smaller pieces. Other catheters mechanically capture and remove clot without thrombolytic agents, thereby reducing the bleeding risk incurred by these drugs. An early example of this is the Fogarty Balloon Thrombectomy catheter. More recent examples include the ClotTriever® (Inari Medical, Irvine, CA) and the ReVene® Thrombectomy Catheter (Vetex Medical, Galway, Ireland).

Unfortunately, many of these therapies have limited success for partial or full blockages caused by chronic thrombus (i.e., a thrombus over one or two months old). As the clot remains in the limb over a period of months, the initial thrombus transforms acutely into a fibrin structure, and chronically can become a collagen structure which is tougher and more firmly adhered to the wall. Chronic thrombus may take the form of fibrous trabeculae or membranes stretching into and across the vein lumen (also known as venous synechiae). Further, the thrombus becomes more firmly attached to the wall. Catheter-based thrombolysis or thrombectomy devices have a lower success rate in removing these blockages. Venous synechiae may also prevent optimal treatment of venous obstruction by balloon angioplasty or stenting, as the fibrous structures prevent permanent stretching of the vessel wall. There is a need for an improved endovascular thrombectomy device which is able to successfully remove chronic thrombus.

### SUMMARY

The subject technology is illustrated, for example, according to various aspects described below, including with reference to FIGS. 1A-11. Various examples of aspects of the subject technology are described as numbered examples (1, 2, 3, etc.) for convenience. These are provided as examples and do not limit the subject technology.
1. A device for capturing material in a blood vessel, the device comprising:
an elongate control member having a proximal portion and a distal portion, the distal portion configured to be intravascularly at a treatment site in a blood vessel lumen proximate an obstruction;
an expandable basket coupled to the distal portion of the control member with the control member extending longitudinally through the basket, the basket comprising a plurality of braided filaments defining an interior cavity, wherein the basket has a closed distal end portion and an opening defined by a proximal edge of the basket, the opening being in communication with the interior cavity; and
first and second legs having respective proximal ends coupled to the distal portion of the control member at a connection and distal ends at the proximal edge of the basket, wherein each of the first and second legs are formed of bundled portions of the filaments,
wherein the first and second legs are configured to position the basket within the blood vessel lumen independent of the path of the control member such that the proximal edge of the basket remains in contact with an inner surface of the blood vessel lumen when the basket is positioned around a curve.
2. A device for capturing material in a blood vessel, the device comprising:
an elongate control member having a proximal portion and a distal portion, the distal portion configured to be intravascularly at a treatment site in a blood vessel lumen proximate an obstruction;
an expandable basket coupled to the distal portion of the control member with the control member extending longitudinally through the basket, the basket comprising a plurality of braided filaments defining an interior cavity, wherein the basket has a closed distal end portion and an opening defined by a proximal edge of the basket, the opening being in communication with the interior cavity; and
first and second legs having respective proximal ends coupled to the distal portion of the control member at a connection and distal ends at the proximal edge of the basket, wherein each of the first and second legs are formed of bundled portions of the filaments,
wherein the first and second legs space the proximal edge of the basket longitudinally from the connection to the control member, thereby mechanically decoupling the proximal edge from the control member such that, when the basket is positioned around a curve, the proximal edge remains in contact with an inner surface of the blood vessel.
3. The device of Example 1 or Example 2, wherein the bundled filaments of the first and second legs branch at distal ends of the first and second legs into respective upper and lower arms, wherein each of the upper arms and each of the lower arms are formed of bundled filaments.
4. The device of Example 3, wherein the filaments branch distally away from the filament bundle of the respective upper and lower arms to form the sidewall of the basket.
5. The device of Example 4, wherein:
the first leg splits at its distal end into a first upper arm and a first lower arm, the first upper arm and first lower arm defining a first prong, and
the second leg splits at its distal end into a second upper arm and a second lower arm, the second upper arm and second lower arm defining a second prong.
6. The device of Example 5, wherein the first and second upper arms converge along a circumferential direction towards one another as the first and second upper arms extend distally, and wherein the distal ends of the first and second upper arms are spaced apart from one another along a circumferential direction by a gap.
7. The device of Example 6, wherein the basket includes a reinforcing element extending along the first and second upper arms, with the bundled filaments, and spanning the circumferential gap between the distal ends of the first and second upper arms.
8. The device of any one of Examples 5 to 7, wherein the first and second lower arms converge along a circumferential direction towards one another as the first and second lower arms extend distally, and wherein the distal ends of the first and second lower arms are spaced apart from one another along a circumferential direction by a gap.
9. The device of Example 8, wherein the basket includes a reinforcing element extending along the first and second lower arms, with the bundled filaments, and spanning the circumferential gap between the distal ends of the first and second lower arms.
10. The device of any one of Examples 5 to 9, wherein the first and second prongs are diametrically opposed.
11. The device of any one of Examples 5 to 10, wherein a sidewall of the basket has a first region comprising the first and second prongs and a second region extending distally from the first and second prongs, and wherein the sidewall is circumferentially continuous along the second region, with the exception of the openings between the interwoven filaments.
12. The device of Example 11, wherein the first and second prongs have a first average pore size and the second region has a second average pore size less than the first average pore size.
13. The device of Example 11 or Example 12, wherein the first and second prongs have a first pic count and the second region has a second pic count greater than the first pic count.
14. The device of any one of Examples 11 to 13, wherein the first and second prongs have a first longitudinal stiffness and the second region has a second longitudinal stiffness that is less than the first longitudinal stiffness.
15. The device of any one of Examples 1 to 14, wherein the filaments do not diverge away from the bundle along the legs.
16. The device of any one of Examples 1 to 15, wherein the basket comprises a proximal structure and a distal structure coupled to the proximal structure at a circumferential joint.
17. The device of Example 16, wherein:
the filaments are first filaments and the proximal structure is formed of the first filaments, and
the distal structure is formed of a plurality of interwoven second filaments.
18. A device for capturing material in a blood vessel, the device comprising:
a first structure comprising a first braid formed of a plurality of interwoven first filaments, the first structure having a first radial strength and configured to press radially outwardly against a vessel wall to anchor the device within a vessel;
a second structure comprising a second braid formed a plurality of interwoven second filaments, the second structure having a second radial strength less than the first radial strength, wherein the second structure is configured to capture and retain liberated portions of obstructive material in the vessel lumen;
a joint between the first structure and the second structure, wherein, at the joint, the first filaments extend through openings defined by the second filaments and bend back proximally, thereby coupling the first structure to the second structure.
19. A device for capturing material in a blood vessel, the device comprising:
an elongate control member having a proximal portion and a distal portion, the distal portion configured to be intravascularly at a treatment site in a blood vessel lumen proximate an obstruction;
an expandable basket coupled to the distal portion of the control member with the control member extending longitudinally through the basket, the basket comprising a plurality of braided filaments defining an interior cavity, wherein the basket has a closed distal end portion and an opening defined by a proximal edge of the basket, the opening being in communication with the interior cavity; and
first and second arms having respective proximal ends coupled to the distal portion of the control member, wherein each of the first and second arms are formed of bundled portions of the filaments and define a majority of the proximal edge of the basket,
wherein the first and second arms converge along a circumferential direction towards one another as the first and second arms extend distally, and wherein the distal ends of the first and second arms are spaced apart from one another along a circumferential direction by a gap.
20. The device of Example 19, wherein the bundled filaments branch away from the respective first and second arms to form a sidewall of the basket.
21. The device of Example 19 or Example 20, wherein the proximal edge of the basket comprises only the filaments and does not include a reinforcing element.
22. The device of Example 19 or Example 20, wherein the basket includes a reinforcing element extending along the first and second arms, with the bundled filaments, and spanning the circumferential gap between the distal ends of the first and second arms.
23. A method, comprising:
advancing an elongate shaft over an elongate member such that that the elongate member is disposed within a lumen of the elongate shaft, wherein a proximal portion of the elongate shaft comprises a coupling portion and a coupler coupled to the coupling portion; and
while the elongate member is positioned within a lumen of the coupler and the lumen of the elongate shaft along the coupling portion, moving the coupler axially relative to the coupling portion, thereby temporarily securing the axial positions of the elongate shaft and the elongate member relative to one another.
24. The method of Example 23, wherein the elongate shaft is a first elongate shaft and the method further comprises advancing a second elongate shaft over the elongate member, the elongate shaft, and the coupler while the coupler is temporarily securing the elongate shaft to the elongate member.
25. The method of Example 23 or Example 24, wherein moving the coupler axially occurs in a first direction and wherein the method further comprises decoupling the elongate shaft from the elongate member by moving the coupler axially in a second direction opposite the first direction.
26. The method of any one of Examples 23 to 25, wherein moving the coupler axially to temporarily secure the elongate shaft to the elongate member comprises sliding the coupler proximally relative to the elongate shaft.
27. The method of any one of Examples 23 to 26, wherein moving the coupler axially to decouple the elongate shaft and the elongate member comprises sliding the coupler distally relative to the elongate shaft.
28. The method of any one of Examples 23 to 27, wherein the elongate member is guidewire.
29. The method of any one of Examples 23 to 28, wherein the elongate member comprises an elongate tubular structure defining a lumen.
30. The method of any one of Examples 23 to 29, further comprising advancing the elongate member to a treatment site within the vasculature prior to advancing the elongate shaft over the elongate member.
31. The method of any one of Examples 23 to 30, wherein advancing the elongate shaft over the elongate member includes advancing a distal end of the elongate shaft over a proximal end of the elongate member.
32. The method of any one of Examples 23 to 31, wherein the coupler comprises a tubular structure defining a lumen extending therethrough.
33. The method of Example 32, wherein the coupler is disposed around an outer surface of the coupling portion of the elongate shaft such that the coupling portion is disposed within the lumen of the coupler.
34. The method of Example 32 or Example 33, wherein the coupler has a first length along which the lumen has a substantially constant diameter and a second length along which the lumen has a diameter that tapers.
35. The method of Example 34, wherein the first length is distal of the second length.
36. The method of any one of Examples 32 to 35, wherein the coupler lumen tapers in a proximal direction.
37. The method of any one of Examples 32 to 36, wherein moving the coupler axially in a first direction forces the coupling portion of the elongate shaft into the tapered portion of the coupler lumen such that an inner surface defining the coupler lumen presses the coupling portion radially inwardly, onto an outer surface of the elongate member, thereby forming an interference fit between the elongate shaft and the elongate member.
38. The method of any one of Examples 23 to 37, wherein the coupling portion comprises the distalmost portion of the elongate shaft.
39. The method of any one of Examples 23 to 38, wherein the coupling portion comprises two proximally extending prongs.
40. The method of any one of Examples 23 to 39, wherein the coupling portion comprises a means for limiting axially movement of the coupler relative to the elongate shaft.
41. The method of any one of Examples 23 to 40, wherein the coupling portion includes a longitudinally extending slot defined by a sidewall of the elongate shaft long the coupling portion.
42. The method of Example 41, wherein an inner surface of the coupler includes a protrusion extending radially inwardly, and wherein, when the coupler is coupled to the coupling portion, the protrusion is slidably disposed within the slot of the coupling portion.
43. The method of Example 42, wherein the coupler includes a waist portion, and wherein, when the coupler is coupled to the coupling portion, the waist portion is slidably disposed within the slot of the coupling portion.
44. The method of any one of Examples 23 to 43, wherein the coupling portion includes a stop element disposed around an outer surface of the elongate shaft at the coupling portion.
45. The method of any one of Examples 23 to 43 or 44, wherein the coupler includes a stop element disposed around an inner surface of the coupler.
46. The method of any one of Examples 23 to 43, wherein:
the coupling portion includes a first stop element disposed around an outer surface of the elongate shaft at the coupling portion,
the coupler includes a second stop element disposed around an inner surface of the coupler, and
when the coupler is coupled to the coupling portion, the first stop is proximal of the second stop.
47. A system, comprising:
an elongate shaft having a proximal portion configured to be positioned at an extracorporeal position and a distal portion configured to be intravascularly delivered to a treatment site within a blood vessel lumen, wherein the elongate shaft comprises a tubular structure defining a first lumen configured to slidably receive a guidewire therethrough, and wherein the proximal portion of the elongate shaft includes a coupling portion; and
a coupler slidably coupled to the coupling portion and extending proximally beyond a proximal end of the elongate shaft, wherein the coupler comprises a tubular structure defining a second lumen configured to receive the guidewire therein,
wherein, when the guidewire is positioned within the first and second lumens, axial movement of the coupler relative to the coupling portion temporarily secures the elongate shaft to the guidewire.
48. The system of Example 47, further comprising a handle configured to be detachably coupled to the coupler such that movement of the handle causes a corresponding movement of the coupler.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A shows a treatment system in accordance with several embodiments of the present technology.

FIG. 1B shows the treatment system of FIG. 1A with the capture device delivered through the cutting device in accordance with several embodiments of the present technology.

FIG. 2A is a perspective view of a coupler coupled to a proximal portion of the treatment system, in accordance with several embodiments of the present technology.

FIG. 2B shows the assembly of FIG. 2A but with the coupler removed for ease of viewing the underlying structures.

FIG. 2C is a cross-sectional side view of the assembly shown in FIG. 2A but with the guidewire removed for ease of viewing the underlying structures.

FIG. 2D is an enlarged view of a portion of the assembly shown in FIG. 2C.

FIG. 3A is a perspective view of a coupler coupled to a guidewire and a shaft, in accordance with several embodiments of the present technology.

FIG. 3B shows the assembly of FIG. 3A but with the coupler removed for ease of viewing the underlying structures.

FIG. 3C is a cross-sectional side view of a portion of the assembly shown in FIG. 3A.

FIG. 4A shows a coupler configured in accordance with several embodiments of the present technology.

FIG. 4B shows the assembly of FIG. 4A but with the coupler removed for ease of viewing the underlying structures.

FIG. 4C is a cross-sectional side view of the assembly shown in FIG. 4A but with the guidewire removed for ease of viewing the underlying structures.

FIG. 4D shows a handle for use with a coupler in accordance with several embodiments of the present technology.

FIGS. 5A-5D show a method of treating a patient using the treatment systems in accordance with several embodiments of the present technology.

FIG. 6A is a perspective view of a mesh basket in accordance with several embodiments of the present technology.

FIG. 6B is a side view of the mesh basket of FIG. 6A, in accordance with several embodiments of the present technology.

FIG. 6C is a top view of the mesh basket of FIGS. 6A and 6B, in accordance with several embodiments of the present technology.

FIG. 6D is an enlarged view of a joint of the mesh basket of FIGS. 6A-6C, in accordance with several embodiments of the present technology. The mesh basket is shown with an opaque cylindrical body positioned therein for the purpose of better visualizing the individual filaments at a top aspect of the mesh basket.

FIG. 6E is an enlarged view of a mouth of the mesh basket of FIGS. 6A-6C, in accordance with several embodiments of the present technology.

FIG. 7 is an enlarged view of a mouth of a mesh basket in accordance with several embodiments of the present technology. The mesh basket is shown with an opaque cylindrical body positioned therein for the purpose of better visualizing the individual filaments at a top aspect of the mesh basket.

FIG. 8 is an enlarged view of a mouth of a mesh basket in accordance with several embodiments of the present technology. The mesh basket is shown with an opaque cylindrical body positioned therein for the purpose of better visualizing the individual filaments at a top aspect of the mesh basket.

FIG. 9 is a top view of a proximal edge configuration for a mesh basket in accordance with several embodiments of the present technology.

FIG. 10A is a perspective view of a mesh basket in accordance with several embodiments of the present technology.

FIG. 10B is a side view of the mesh basket of FIG. 10A, in accordance with several embodiments of the present technology.

FIG. 11 shows the mesh basket of FIGS. 10A and 10B positioned in a curved tube, in accordance with several embodiments of the present technology.

### DETAILED DESCRIPTION

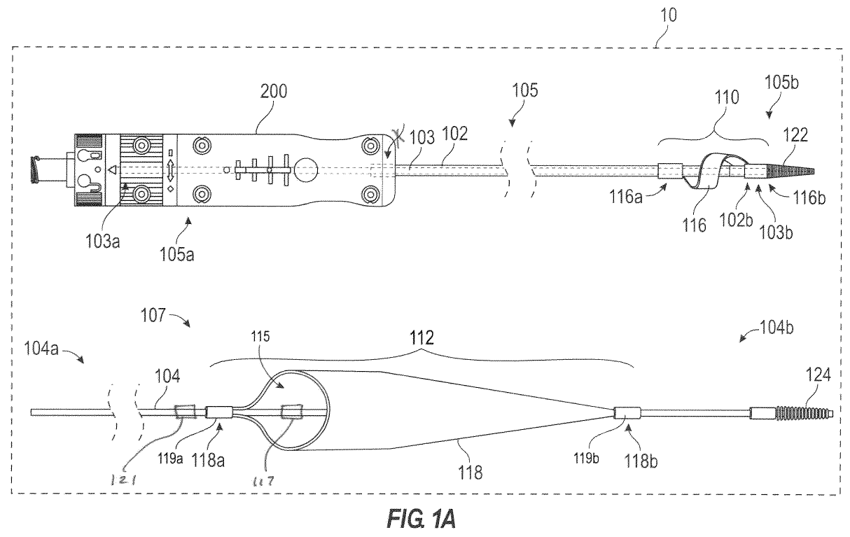

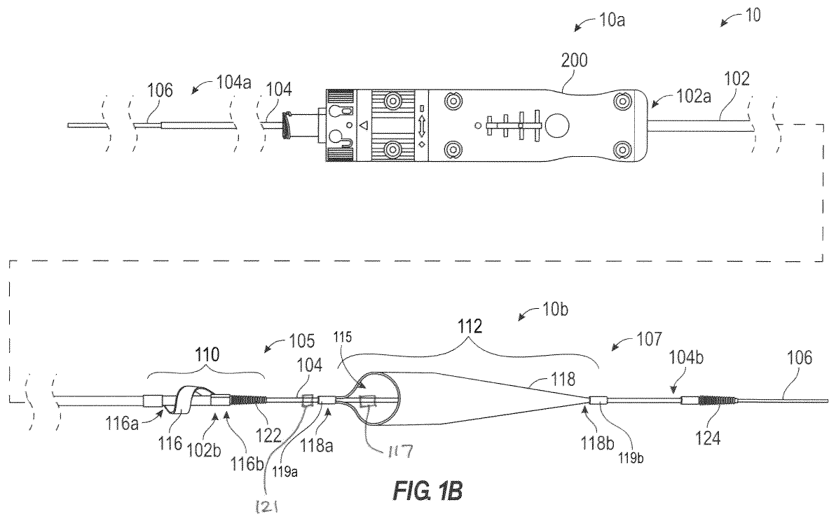

The present technology relates to endovascular treatment systems and associated devices and methods. Some embodiments of the present technology, for example, are directed to mesh baskets for the capture and removal of obstructive material from blood vessel lumens. The mesh baskets disclosed herein include a proximal mouth through which obstructive material enters the basket for capture. The mouth is configured to press radially outwardly against the vessel wall to form a seal that prevents liberated material from bypassing the basket. Given the highly elastic and compressible nature of veins, the basket is also configured to seal in non-uniform vessel geometries including significantly compressed (e.g., flattened) lumens. The embolic collection baskets of the present technology can be particularly well-suited for use with mechanical thrombectomy devices for treating DVT or post thrombotic syndrome (PTS), although other endovascular applications are possible. Specific details of several embodiments of the technology are described below with reference to FIGS. 1A-11.

### I. Treatment Systems and Associated Devices and Methods

FIG. 1A schematically depicts a treatment system 10 (also referred to herein as “the system 10”) configured in accordance with the present technology. The treatment system 100 is configured to access a body lumen (such as a vein or artery) and modify, capture, and/or remove obstructive material from the body lumen at a treatment site. As used herein, “obstruction” or “obstructive material” can comprise, for example, clot material, atherosclerotic plaque, and/or other flow-obstructing structures, including those derivative of clot material, such as fibrotic clot material, venous synechiae, fibrinous structures, collagenous structures, fibrous trabeculae, and/or others. As shown in FIG. 1A, the system 10 can comprise a cutting device 105 and a capture device 107, each configured to access the treatment site. In other embodiments, the system 10 comprises only the cutting device 105 or only the capture device 107.

The cutting device 105 has a proximal portion 105a configured to be positioned extracorporeally during the procedure, a distal portion 105b configured to be positioned at the treatment site within the body lumen, a handle 200 at the proximal portion 105a, and a cutting assembly 110 at the distal portion 100b. The device 100 further includes an outer shaft 102 extending from a proximal portion 102a at or within the handle 200 to a distal portion 102b at the cutting assembly 110, and an inner shaft 103 extending from a proximal portion 103a at or proximal of the handle 200 to a distal portion 103b at the cutting assembly 110. The outer shaft 102 can comprise an elongate tubular structure defining a lumen, and the inner shaft 103 may be disposed within the outer shafter 102. The inner shaft 103 can comprise an elongate tubular structure defining a lumen configured to be slidably positioned over a guidewire 106 (see FIG. 1B) or other device. The lumen of the inner shaft 103, for example, can be configured to accommodate a guidewire having a diameter of from about 0.010 inches to about 0.070 inches, about 0.010 inches to about 0.040 inches, about 0.030 inches to about 0.060 inches, or at least about 0.010 inches, or no more than about 0.038 inches, or about 0.035 inches, about 0.060 inches, etc.

In some embodiments, the cutting device 105 optionally includes a nosecone 122 disposed at a distal portion of the inner or outer shafts 102, 103 distal of the cutting assembly 110. The nosecone 122 can have a tapered profile and/or a spiraling outer surface topography configured to facilitate engagement and/or penetration of obstructive material. The nosecone 122 can be any of the nosecones described in PCT Application No. PCT/US24/19465, filed Mar. 11, 2024, titled INTERVENTIONAL SYSTEMS AND ASSOCIATED DEVICES AND METHODS, which is incorporated herein by reference in its entirety.

The cutting assembly 110 can comprise one or more cutting elements configured to cut through obstructive material in the vessel lumen, thereby separating and/or releasing obstructive material from the vessel wall, a non-native structure positioned within the vessel (such as a failed stent), and/or from other obstructive material. In the example shown in FIG. 1, the cutting assembly 110 comprises a cutting element 116 comprising a helical ribbon having a proximal end 116a fixed to the outer shaft 102 and a distal end 116b fixed to the inner shaft 103. The outer and inner shafts 102, 103 are rotated relative to one another to radially expand and collapse the helical ribbon 116 relative to a longitudinal axis of the cutting device 105. During expansion and collapse, the proximal and distal ends 116a, 116b of the cutting element 116 do not move relative to one another. While a helical ribbon cutting element 116 is shown in FIGS. 1A and 1B, the handles and drive handles of the present technology can be used with cutting devices 105 having other cutting elements and/or cutting assemblies. For example, additional description of the cutting assembly 110, as well as additional cutting assemblies (also referred to as “cutting portions”) can be found in, for example, U.S. Patent Application Publication No. 2024/0074784, filed Mar. 10, 2023, which is disclosed by reference herein in its entirety.

In some embodiments, the handle 200 of the treatment system 10 can be configured to enable controlled rotation of the outer and inner shafts 102, 103 of the cutting device 105 relative to one another to expand and collapse the helical cutting element 116 (if such a cutting element 116 is used). The handle 200 can be any of the handles described in PCT Application No. PCT/US24/19463, filed Mar. 11, 2024, titled ENDOVASCULAR MEDICAL DEVICES AND ASSOCIATED DEVICES, SYSTEMS, AND METHODS, which is incorporated herein by reference in its entirety.

Referring still to FIG. 1A, the capture device 107 comprises a shaft 104 having a proximal portion 104a configured to be positioned extracorporeally during the procedure and a distal portion 104b configured to be positioned at the treatment site within the body lumen. The capture device 107 further includes a capture assembly 112 carried by the distal portion 104b of the shaft 104. The shaft 104 can comprise an elongate tubular structure defining a lumen that is configured to slidably receive a guidewire 106 (see FIG. 1B) or other device therethrough. The lumen of the shaft 104, for example, can be configured to accommodate a guidewire having a diameter of from about 0.010 inches to about 0.038 inches, or at least about 0.010 inches, or no more than about 0.038 inches, or about 0.035 inches, etc.

In some embodiments, the capture device 107 optionally includes a nosecone 124 disposed at a distal portion of the shaft 104, distal of the capture assembly 112. The nosecone 124 can have a tapered profile and/or a spiraling outer surface topography configured to facilitate engagement and/or penetration of obstructive material. The nosecone 124 can be any of the nosecones described in PCT Application No. PCT/US24/19465, filed Mar. 11, 2024, titled INTERVENTIONAL SYSTEMS AND ASSOCIATED DEVICES AND METHODS, which is incorporated herein by reference in its entirety.

The capture assembly 112 can comprise one or more expandable mesh structures configured to engage, trap, or otherwise become enmeshed with obstructive material at the treatment site before, during, or after engagement by the cutting assembly 112. In some embodiments, for example as shown in FIG. 1A, the capture assembly 112 can comprise a mesh basket 118 (or “basket 118”) having proximal and distal end portions 118a, 118b coupled to the shaft 104 at proximal and distal hubs 119a, 119b, respectively. The mesh basket 118 can have a closed distal region and an opening or mouth 115 at the proximal region. A sidewall of the basket 118 defines an interior cavity in communication with the mouth 115.

The basket 118 can have a collapsed or constrained configuration for delivery to the treatment site through a sheath, and an expanded configuration once removed from the constraints of the sheath. The collapsed or constrained diameter of the basket 118 may be of from about 3 mm to about 6 mm. In the collapsed diameter, the basket 118 can be configured to be delivered through a sheath having a diameter no greater than 10 Fr. The mesh basket 118 can also be configured to be removed through a sheath having an inner diameter of from about 9 Fr to about 16 Fr, or about 12 Fr to about 16 Fr. The expanded, unconstrained diameter of the basket 118 may be determined by the intended treatment location so that the basket 118 achieves adequate vessel wall contact during use. For example, for those embodiments in which the basket 118 is used in the inferior vena cava (IVC), the basket 118 can have an expanded, unconstrained diameter of at least 28, 29, or 30 mm. As another example, for those embodiments in which the basket 118 is used in the iliac through femoral/popliteal vein segments, the basket 118 can have an expanded, unconstrained diameter of at least 16, 17, or 18 mm. It will be appreciated that other treatment locations and basket diameters are possible. In any case, the unconstrained, expanded diameter may be oversized (up to 10%) relative to the native vessel diameter.

The proximal end portion 118a of the basket 118 may be axially fixed to the shaft 104 or may be free to slide along the shaft 104. Likewise, the proximal end portion 118a may be rotationally fixed to the shaft 104 or may be free to rotate about the shaft 104. The distal end portion 118b of the basket 118 may be axially fixed to the shaft 104 or may be free to slide along the shaft 104. Allowing the distal end portion 118b to translate along the shaft 104 can be beneficial for allowing foreshortening (e.g., changes in length) of the basket 118 during deployment. The distal end portion 118b may be rotationally fixed to the shaft 104 or may be free to rotate about the shaft 104. Allowing rotation of the distal end portion 118b can facilitate adjustment of the basket 118 to an uneven radial distribution of obstructive material within its interior cavity, especially during proximal withdrawal and resheathing of the basket 118. In some examples, the proximal end portion 118a is rotationally and axially fixed relative to the shaft 104 while the distal end portion 118b is free to rotate and translate relative to the shaft 104. In other embodiments, the proximal end portion 118a is axially fixed along the shaft 104 but free to rotate while the distal end portion 118b is free to rotate and translate relative to the shaft 104. Allowing rotation of both the proximal and distal end portions 118a, 118b enables rotation of the shaft 104 (e.g., to engage the obstructive material with the nosecone 124) without also rotating the basket 118, which can remain relatively rotationally stationary, along with the sheath. Forcing the basket 118 to rotate with the shaft 104 while the sheath remains stationary can twist the basket 118 within the sheath, which can make deployment of the basket 118 more difficult.

In some embodiments, the capture assembly 112 may include one or more stops positioned at various locations along the shaft 104. For example, the capture assembly 112 can optionally include a stop 121 positioned proximal of the proximal hub 119a. Additionally or alternatively, the capture assembly 112 can optionally include a stop 117 positioned between the proximal and distal hubs 119a, 119b. The stop 117 can be fixed relative to the shaft 104, and at least in those embodiments in which the distal hub 119b is configured to slide axially along the shaft 104, the stop 117 prevents axial movement of the distal hub 119b proximally beyond the stop 117. Likewise, in those embodiments in which the proximal hub 119a is slidable along the shaft 104, stop 117 would prevent axial movement of the proximal hub 119a distally along the shaft 104. Stop 121 may limit axial movement of the proximal hub 119a proximally of the stop 121. In many examples the capture assembly 112 does not include stop 121 and/or stop 117.

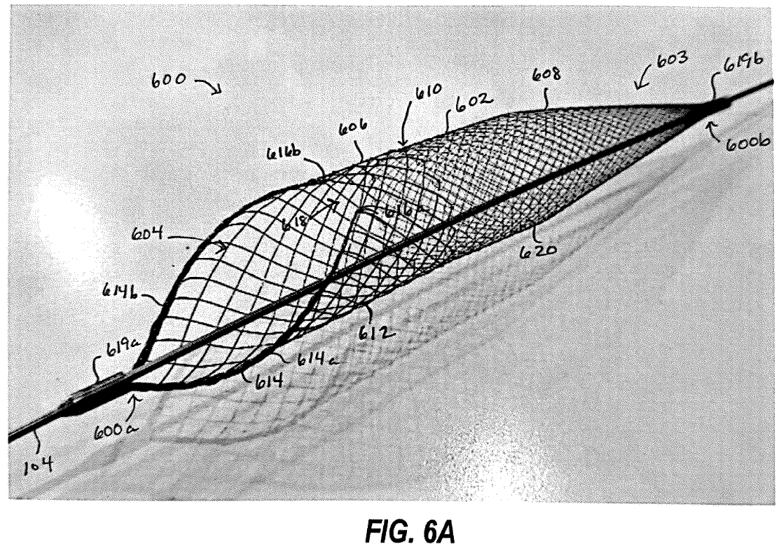

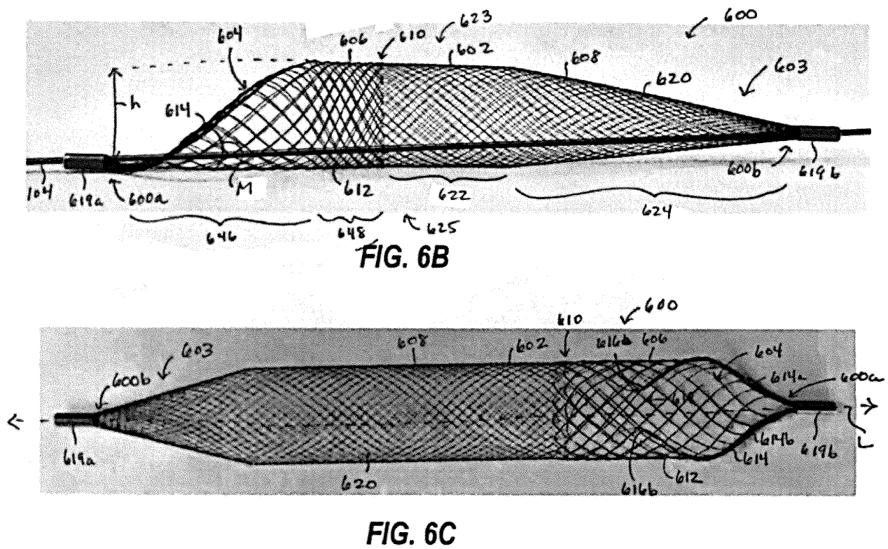

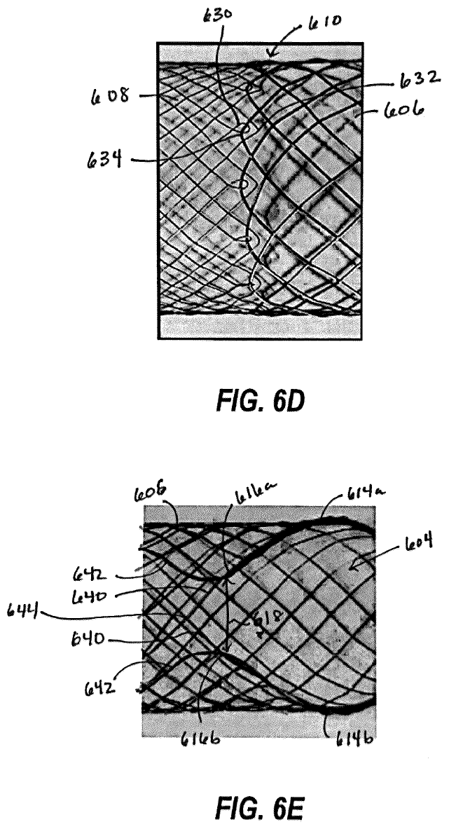

When the cutting device 105 is coupled to the capture device 107 (see FIG. 1B), the basket 118 can be distal of the cutting assembly 110 such that obstructive material separated from the treatment site by the cutting assembly 110 can be collected by the basket 118, either by pulling the basket 118 proximally during the procedure or due to the basket 118 being downstream from the cutting assembly 110. Additional description of the capture assembly 112 is provided below with reference to FIGS. 6A-10.

FIG. 1B shows the treatment system 10 arranged such that the cutting device 105 is positioned over the capture device 107. In such an arrangement, the shaft 104 of the capture device 107 is disposed within the lumen of the inner shaft 103 of the cutting device 105, which is positioned over a guidewire 106. The shaft 104 can extend proximally through the handle 200 of the cutting device 105 until exiting the proximal end of the handle 200. In some cases, the handle 200 can include a luer lock coupled to a proximal end of the handle 200, and the shaft 104 can extend through the luer lock.

In use, the guidewire 106 can be advanced through the vasculature to a treatment site (e.g., the site of an occlusion or other vascular disease being treated), and a guide sheath (not shown) can be delivered over the guidewire 106 until a distal end of the guide sheath is positioned proximal of the treatment site. A delivery sheath (not shown) containing the capture device 107 in a collapsed and/or compressed state can be advanced through the guide sheath, over the guidewire 106, until a distal end of the delivery sheath is proximate the treatment site. In those cases in which the treatment system 10 is configured to treat obstructed lumens, the distal end of the delivery sheath can be positioned distal of the obstruction.

The basket 118 can be deployed from the distal end of the delivery sheath such that the basket 118 expands and the proximal edge surrounding the mouth 115 presses radially outwardly against the inner surface of the vessel wall, thereby forming a seal against the wall. The cutting device 105 can then be advanced over the capture device 107 (e.g., such that the inner shaft 103 of the cutting device 105 is advanced over the shaft 104 of the capture device 107) to the treatment site. The cutting device 105 can then be actuated to cut, separate, and/or otherwise modify the obstruction to recanalize the vessel. During the debulking procedure, liberated material may flow downstream into the mesh basket 118. When the obstruction is sufficiently debulked, the cutting device 105 can be removed from the patient. The mesh basket 118 and its contents are then pulled proximally into the guide sheath for removal from the body.

The treatment systems and methods of the present technology can be used to treat a variety of vascular conditions. Non-limiting examples include treatment of lower extremity deep venous obstruction, including clearing of DVT, chronically occluded iliofemoral stents, chronic wall adherent native vessel obstruction, obstructed femoral and popliteal inflow veins, and/or trabeculae and/or synechia webbing from the vasculature. Specifically with respect to recanalization procedures, the treatment systems and methods can be used to recanalize in-stent restenosis (ISR), post thrombotic tissue adjunctive to venous stenting, residual (“late-DVT”) adjunctive to pharma-mechanical thrombectomy, and in-flow vessel obstruction (e.g., femoral/popliteal). Other interventional applications are possible.

Additional details regarding various methods of using the treatment devices of the present technology are disclosed elsewhere herein.

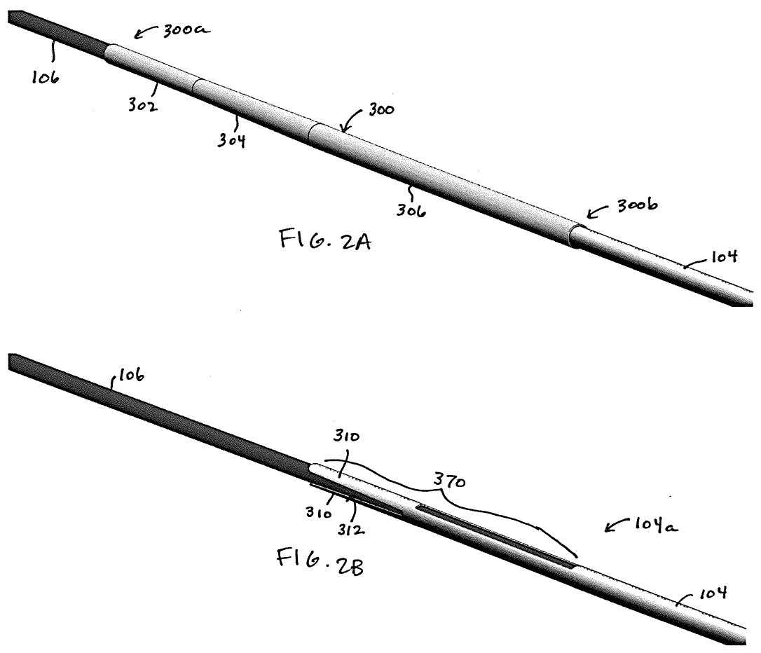

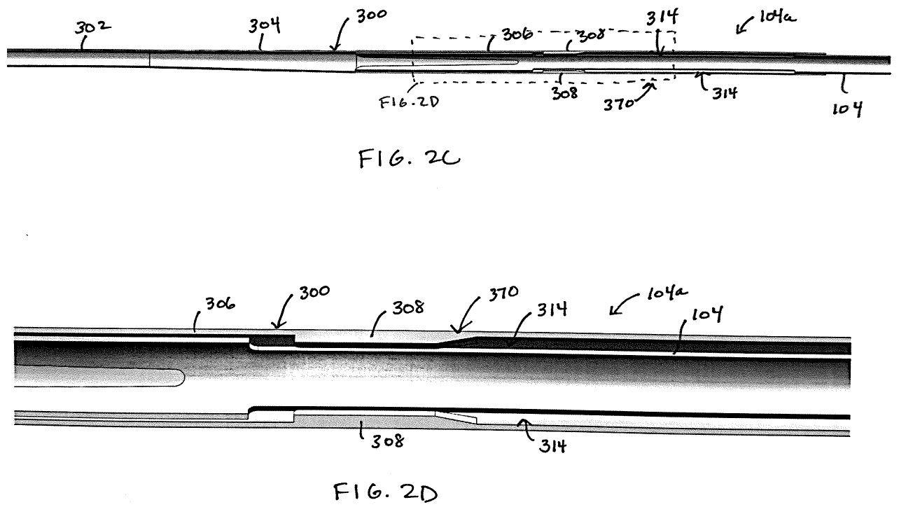

In some embodiments, the capture device 107 optionally includes a coupler at a proximal portion 104a of the shaft 104 that is configured to detachably fix a proximal portion of the guidewire 106 to a proximal portion of the shaft 104 once the shaft 104 has been advanced to a desired location. The coupler can be configured to secure the guidewire 106 and shaft 104 axially relative to one another, in some cases rotationally as well axially. The coupler can be low-profile (e.g., having an outer diameter of about 0.010 inches to about 0.100 inches, no more than 0.010 inches, no more than 0.012 inches, no more than 0.020 inches, no more than 0.030 inches, no more than 0.040 inches, no more than 0.050 inches, no more than 0.060 inches, no more than 0.070 inches, or no more than 0.100 inches) such that another interventional device can be advanced over the guidewire 106, shaft 104, and coupler. The low-profile coupler, for example, may increase the overall diameter of the proximal portion of the shaft 104 by no more than about 0.015 inches, or by no more than about 0.010 inches. The cutting device 105 of the present technology can be delivered over the guidewire 106, shaft 104, and coupler by sliding the inner shaft 103 over all three components. Conventional mechanisms for axially coupling a guidewire to a shaft of an interventional device typically comprise a larger profile device (e.g., a luer lock or other hub) that cannot be received within the lumen of any other interventional device. As such, any other interventional instruments required for the procedure must either be used at a separate time (which, in the case of embolic collection, would not have value) or integrated into a single device, which presents a host of engineering and practical challenges. The couplers of the present technology beneficially enable delivery of other interventional devices over the capture device 105 (e.g., via backloading) while anchoring the shaft 104 to the guidewire 106 to prevent unintended movement of the mesh basket during device advancement and removal. Other interventional devices include cutting devices (such as cutting device 105), as well as any larger tube, catheter, sheath, etc., whether alone or carrying a treatment element at its distal end. It will be appreciated that the couplers disclosed herein may be used to detachably couple any elongate shaft to a guidewire, and not just shaft 104 carrying a capture assembly 112. Example couplers are detailed below with reference to FIGS. 2A-4C.

FIGS. 2A-2D show an example of a coupler 300 configured in accordance with several embodiments of the present technology. FIG. 2A shows a proximal portion of a guidewire 106, a proximal portion 104a of the shaft 104, and a coupler 300 positioned over both the guidewire 106 and the shaft 104. In FIG. 2B the coupler 300 has been removed to show a coupling portion 370 of the shaft 104. As shown, the coupler 300 may comprise a tubular element defining a lumen therethrough. The coupler 300 has proximal portion 300a and a distal portion 300b, and the coupler 300 is configured to be coupled to the shaft 104 such that coupler 300 extends proximally beyond a proximal end of the shaft 104. As best shown in the cross-sectional side view of the shaft 104 and coupler 300 in FIG. 2C (guidewire 106 removed for ease of viewing the shaft 104 and coupler 300) The coupler 300 includes a first region 302 along which the coupler 300 has a first inner diameter, a second region 304 distal of the first region 302 and along which the coupler 300 has a second inner diameter, and a third region 306 distal of the second region 304. The third inner diameter can be greater than the first inner diameter, with the second inner diameter tapering proximally between the third and first diameters. In some embodiments, the first and third diameters may be substantially uniform along their respective lengths. In other embodiments, one or both of the first and third diameters may vary. The outer diameter of the coupler 300 can mirror that of the inner diameter (e.g., varying) or may be substantially uniform along its length.

The lumen defined by the first region 302 is configured to slidably receive the guidewire 106 therein. While the lumen along the second region 304 is also configured to receive the guidewire 106 therethrough, the second inner diameter tapers proximally from the third inner diameter down to the first inner diameter, which is smaller than the outer diameter of the proximal end portion of the shaft 104 such that the shaft 104 cannot extend through the entire length of the second region 304. The third inner diameter can be substantially constant along the length of the third region 306 and the lumen is configured to receive both the guidewire 106 and the coupling portion 370 of the shaft 104. In some embodiments, the coupler 300 does not include the first region 302 such that the second, tapered region 304 comprises the proximalmost portion of the coupler 300.

As best shown in the cross-sectional side view of the coupler 300 in FIGS. 2C and 2D, the coupler 300 can further include a protrusion 308 extending radially inwardly from the sidewall, into the lumen along the third region 306.

The coupling portion 370 of the shaft 104 can comprise features for limiting axial movement of the coupler 300 relative to the shaft 104 and for engaging with the coupler 300 to lock onto the guidewire 106. For example, in some embodiments the coupler 300 comprises one or more longitudinal slots 314 configured to slidably receive the protrusion 308 of the coupler 300. When engaged, the coupler 300 is thus prevented from proximal and distal axial movement beyond the proximal and distal ends of the slots 314. In some embodiments, the coupler 300 includes a single protrusion 308 and the coupling portion 370 includes a single slot 314. In other embodiments, including as shown in FIGS. 2C-2D, the coupler 300 may include two or more protrusions 308 configured to slide within two or more slots 314 in the shaft 104. In some embodiments, the slot-protrusion engagement prevents relative rotational movement between the shaft 104 and the coupler 300.

The coupling portion 370 of the shaft 104 can further include locking tabs 310 which coincide with the distalmost portion of the shaft 104. Portions of the tube comprising the shaft 104 can be removed (e.g., via laser cutting or other methods) to form the locking tabs 310. As such, each of the locking tabs 310 can be arcuate in cross-section and are separated by gaps 312 in the sidewall. In some embodiments, the locking tabs 310 are diametrically opposed about the shaft sidewall. Other configurations are possible. While FIG. 2B shows a coupling portion 370 including two locking tabs 310, in some embodiments the coupling portion 370 may include more than two locking tabs 310 (e.g., three locking tabs, four locking tabs, etc.).

In an unlocked state, the locking tabs 310 are positioned within the coupler lumen along the third region 306 and the shaft 104 can move axially relative to the guidewire 106. To lock the position of the shaft 104 relative to the guidewire 106, the coupler 300 can be moved distally relative to the shaft 104 (or the shaft 104 moved proximally relative to the coupler 300), thereby forcing the locking tabs 310 into engagement with an inner sidewall of the coupler 300 along the tapering second region 304. The narrowing sidewall presses the locking tabs 310 radially inwardly, against the guidewire 106, to form an interference fit between the shaft 104 and the guidewire 106 and fixing together the axial positions of the guidewire 106 and the shaft 104. To decouple the guidewire 106 and the shaft 104, the coupler 300 can be moved proximally relative to the shaft 104 (or the shaft 104 moved distally relative to the coupler 300), thereby releasing the locking tabs 310.

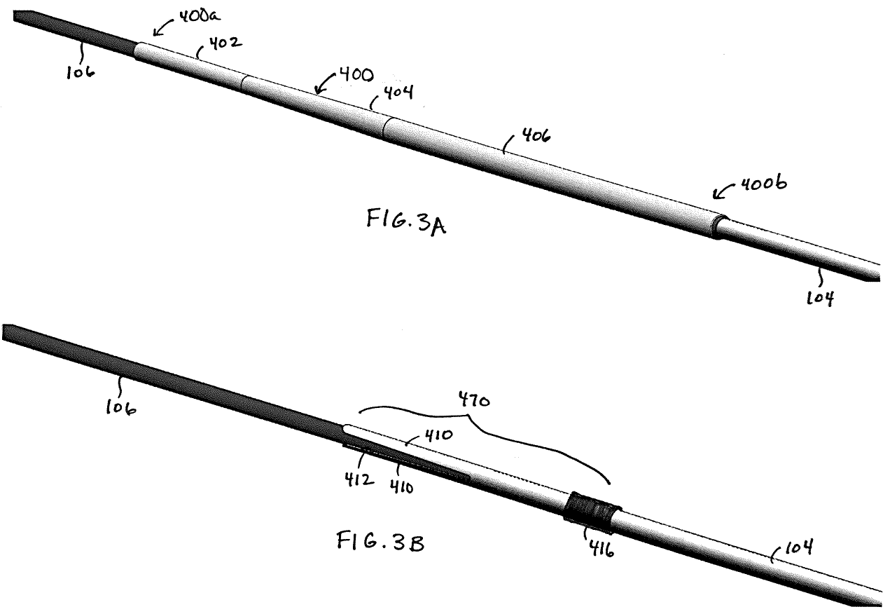

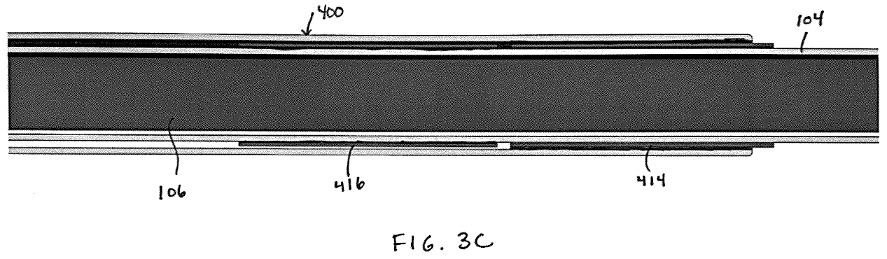

FIGS. 3A-3C show another coupler 400 configured in accordance with several embodiments of the present technology. FIG. 3A is a perspective view of the coupler 400 positioned over the guidewire 106 and the shaft 104, and FIG. 3B shows the assembly of FIG. 3A but with the coupler 400 removed for ease of viewing the underlying structures. FIG. 3C is a cross-sectional side view of a portion of the assembly shown in FIG. 3A. Referring to FIGS. 3A-3C together, the coupler 400 can have first, second, and third regions 402, 404, 406 as described above with reference to FIGS. 2A-2D. The coupler 400 further includes a band 414 disposed in its lumen at the distal end portion 400b of the coupler 400, along the third region 406, effectively decreasing an inner diameter of the coupler 400 at that location. The inner diameter of the band 414 is still configured to slidably receive the shaft 104 therethrough. The band 414 may be a separate component welded onto an inner surface of the coupler 400 or may be integral with the coupler 400 (e.g., a thicker portion of the sidewall).

The coupling portion 470 of the shaft 104 can have features generally similar to the features of the coupling portion 370 shown in FIGS. 2A-2D (e.g., locking tabs 410 are similar to locking tabs 310, gaps 412 are similar to gaps 312, etc.). The coupling portion 470 of FIGS. 3A and 3B further includes a band 416 bonded to an outer surface of the shaft 104, or an otherwise localized portion of the shaft 104 that has a greater outer diameter than the immediately adjacent portions of the shaft 104. When the coupler 400 is positioned over the shaft 104 and guidewire 106, the band 414 of the coupler 400 is positioned distal of the band 416 of the shaft 104. As such, the bands 414, 416 limit proximal movement of the coupler 400 relative to the shaft 104 and distal movement of the shaft 104 relative to the coupler 400. The band 416 may be a separate component welded onto an outer surface of the shaft 104 or may be integral with the shaft 104 (e.g., a thicker portion of the sidewall).

The method of using the coupler 400 for coupling and decoupling of the guidewire 106 and shaft 104 is similar to that described above with reference to FIGS. 2A-2D.

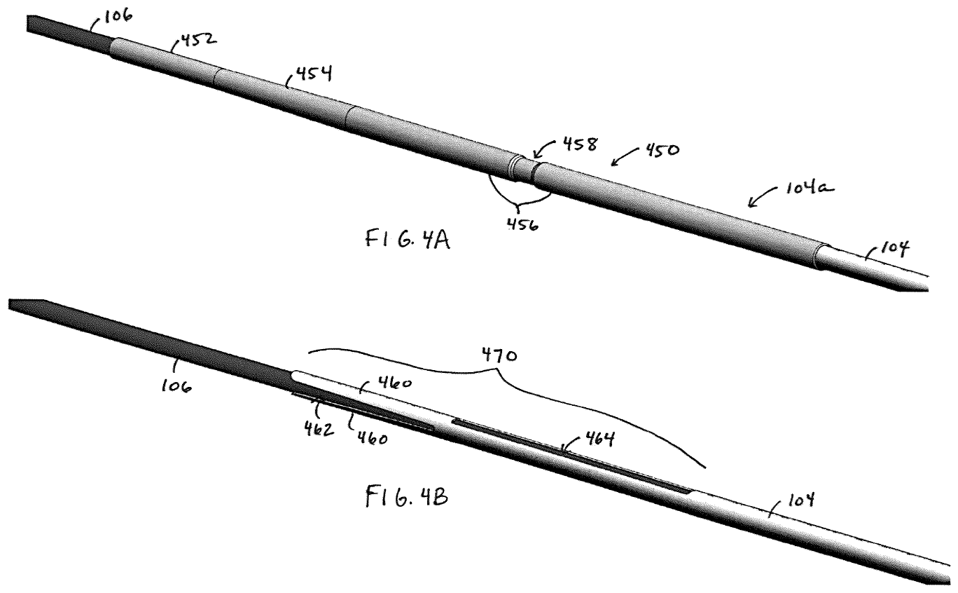

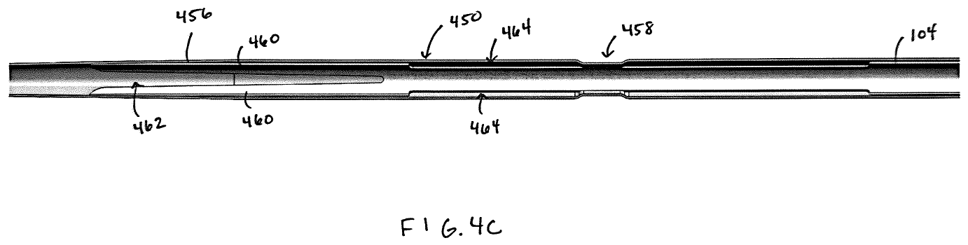

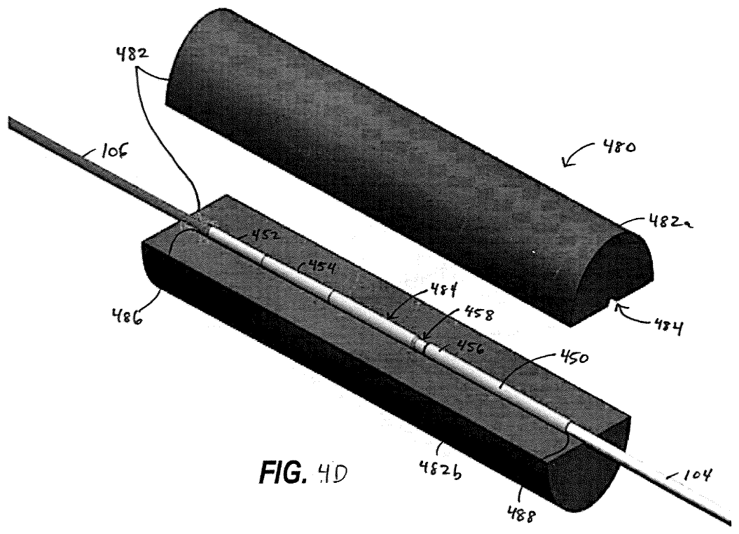

FIGS. 4A-4C show another coupler 450 configured in accordance with several embodiments of the present technology. FIG. 4A shows a proximal portion of a guidewire 106, a proximal portion 104a of the shaft 104, and a coupler 450 positioned over both the guidewire 106 and the shaft 104. In FIG. 4B the coupler 450 has been removed to show a coupling portion 470 of the shaft 104. The coupler 450 can have first, second, and third regions 452, 454, 456 as described above with reference to first, second, and third regions 302, 304, and 306 of FIGS. 2A-2D. The connector 450 can further include a waist portion 458 comprising an annular recess formed along the third region 456. The coupling portion 470 of the shaft 104 can have features generally similar to the features of the coupling portion 470 shown in FIGS. 2A-2D (e.g., locking tabs 410 are similar to locking tabs 460, slots 464 are similar to slots 314, etc.). The coupler 450 is configured to be positioned over the coupling portion 470 of the shaft 104 such that the waist portion 458 is longitudinally aligned with and slides along the slots 464. When engaged, the coupler 450 is thus prevented from proximal and distal axial movement beyond the proximal and distal ends of the slots 464, respectively.

The method of using the coupler 450 for coupling and decoupling of the guidewire 106 and shaft 104 is similar to that described above with reference to FIGS. 2A-2D.

In some examples, the system 10 further includes a handle for gripping and manipulating the coupler (e.g., any of the couplers disclosed herein). For example, the handle may comprise a separate element that is configured to detachably couple to the coupler and that provides increased surface area for improved handling by the user. FIG. 4C depicts one example of such a handle 480, shown with the coupler 450 of FIGS. 4A and 4B received within a portion of the handle 480. It will be appreciated that the handles disclosed herein can be used with any of the couplers disclosed herein.

As shown in FIG. 4C, in some embodiments the handle 480 can comprise a body 482 having a receiving channel 484 configured to receive the coupler therein. In some embodiments, the handle 480 and/or receiving channel 484 is only configured to receive and/or be coupled to the coupler. In other embodiments, for example as shown in FIG. 4C, the handle 480 and/or receiving channel 484 may additionally be configured to couple to the guidewire 106 and/or shaft 104. The body 482 of the handle 480 can comprise two pieces (e.g., a top piece 482a and a separate, detachable bottom piece 482b, configured to be coupled to one another around the coupler 450. In other examples, the body 482 comprises two pieces connected a hinge, or a single piece with a flexible joint that allows rotation of one portion relative to another portion. In yet other embodiments, the body 482 comprises a peel-away grip.

The body 482 can include one or more ledges, steps, grooves, and other features configured to prevent axial movement of the coupler relative to the body. The handle 480 shown in FIG. 4C, for example, has a proximal ledge 486 that engages a proximal end of the coupler 400, and a distal ledge 488 that engages a distal end of the coupler 400. As a result, axial movement of the body 482 (by a user) translates into axial movement of the coupler, thereby enabling control of locking and unlocking the coupler via the handle 480. The handle 480 is completely detachable from the coupler 450 such that it provides the advantages of improved grip without permanently increasing a profile of the coupler, which would not allow for other medical devices to be advanced over the shaft 104.

### II. Example Methods of Use

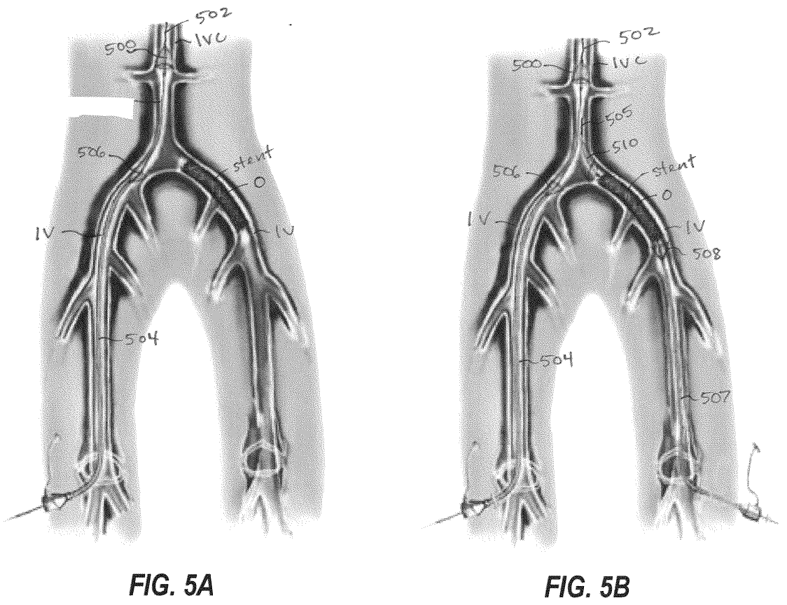

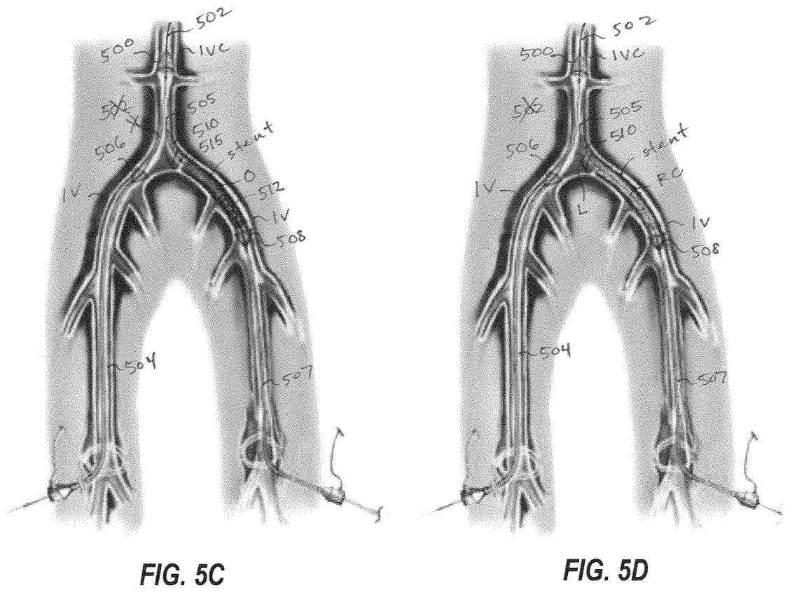

FIGS. 5A-5D illustrate example methods of using the treatment systems disclosed herein to recanalize a chronic venous obstruction and/or remove and/or modify obstructive material within a vein. As shown in FIG. 5A, a patient may present with a chronic venous obstruction, in this case in the iliac vein IV due to in-stent stenosis O. In some cases the method may optionally begin with placement of a secondary mesh basket 500 in the IVC via contralateral delivery (e.g., contralateral to the location of the obstruction O). The secondary mesh basket 500 may comprise any of those disclosed herein, or another. To do deliver the secondary mesh basket 500, a guidewire 502 may be advanced through the vasculature (for example, from an access point in the contralateral femoral vein) to a position within or distal to the IVC. A guide sheath 504 may then be advanced over the guidewire 502 until a distal end of the guide sheath 504 is positioned within the contralateral iliac vein IV. In some examples, a funnel 506 is expanded at the distal end of the sheath 504. A delivery sheath (not shown) containing the secondary mesh basket 500 may then be advanced over the guidewire 502, through the sheath 504 until a distal end of the delivery sheath is disposed within the IVC. The delivery sheath may then be withdrawn, or the secondary mesh basket 500 advanced, to deploy the secondary mesh basket 500 and allow the secondary mesh basket 500 to expand into apposition with the vessel wall. The delivery sheath is then withdrawn proximally from the patient. The secondary mesh basket 500 thus provides a redundant capture mechanism should any liberated material evade the primary mesh basket 510 (see FIG. 5B) and flow into the IVC. In some embodiments, the guide sheath 504 may not include a funnel and/or the funnel may not be deployed, and/or contralateral placement of a secondary mesh basket, or any contralateral device, is not utilized.

As shown in FIG. 5B, a guidewire 505 may be advanced through the vasculature from an ipsilateral location (for example, from an access point in the ipsilateral femoral vein), through the obstruction O, until a distal end of the guidewire 505 is distal of the obstruction O. A guide sheath 507 can then be advanced over the guidewire 505 until the sheath 507 is proximal of, and proximate to, a proximal end of the obstruction O. In some examples, a funnel 508 is optionally expanded at the distal end of the guide sheath 507. In some cases, the funnel 508 is not used. Next, a delivery sheath (not shown) containing a mesh basket 510 (comprising any of the mesh baskets disclosed herein) coupled to a control member (such as shaft 104) can be advanced through the obstruction O until a distal end of the delivery sheath is distal of the obstruction O. The delivery sheath may then be withdrawn, or the basket 510 advanced, to deploy the basket 510 and allow the basket 510 to expand into apposition with the vessel wall. Optionally, the proximal ends of the control member and the guidewire 505 may then be temporarily fixed to one another, for example using any of the couplers disclosed herein. The delivery sheath is then withdrawn proximally from the patient.

As shown in FIG. 5C, a cutting device 512 (comprising any of the cutting devices disclosed herein, or others) can then be advanced through the sheath 507, over the control member 515 of the mesh basket 510. The cutting device 512 is then at least partially expanded and/or rotated to form a channel within the obstruction O. Once a channel is formed, the cutting device 512 can be incrementally expanded and/or rotated to cut and/or break up the obstruction O. During this process, liberated portions L of the obstruction O may flow distally into the mesh basket 510. Once the obstruction O is sufficiently debulked such that normal venous flow is restored through treatment site (see RC in FIG. 5D), the cutting device 512 can be withdrawn proximally through the sheath 507 and removed from the patient. The mesh basket 510, with liberated material L contained therein, can then be withdrawn proximally into the sheath 507 for removal from the patient. In some cases, the ipsilateral funnel 508, if used, can be advanced to a location within the recently-debulked portion of the vessel (including within the stenosed stent, when used in such applications) prior to withdrawal of the mesh basket 510. After withdrawal of the mesh basket 510, the ipsilateral funnel 508 can be collapsed and the ipsilateral sheath 507 and guidewire 505 are withdrawn.

Following removal of the ipsilateral treatment devices, the secondary mesh basket 500 can be withdrawn into the sheath 504. The contralateral funnel 506 can then be collapsed and the contralateral sheath 504 and guidewire 502 are withdrawn.

### III. Example Mesh Baskets

During endovascular treatment of thrombosed or chronically occluded veins of the deep venous system, thrombus or post thrombotic tissue may become liberated from the vessel either intentionally through use of a thrombectomy or debulking device (such as the cutting device disclosed herein) or unintentionally due to manipulation of ancillary endovascular devices within the diseased vessel segment. Once liberated, the embolized material may travel through the bloodstream and obstruct blood flow to the lungs, resulting in severe patient harm or death. The mesh baskets of the present technology may be used perioperatively, either as an integral component to a thrombectomy or post thrombotic debulking device or as a stand-alone temporary distal protection tool, to capture and remove embolized tissue from the vasculature. While the mesh baskets are described herein with reference to debulking and/or venous procedures, it will be appreciated that the mesh baskets may be used for other medical interventions and/or within the arterial system.

As will be explained below, the baskets of the present technology provide several performance advantages over existing devices, including ease of loading into a delivery sheath, ease of deploying from a delivery sheath, ability to consistently provide firm contact/sealing with the vessel wall, and ability to capture and remove liberated tissue through an access or guide sheath.

FIGS. 6A-6E show an example mesh basket 600 (or “basket 600”) for use with the treatment systems of the present technology. The basket 600 has proximal and distal end portions 600a, 600b configured to be coupled to the shaft 104 at proximal and distal hubs 619a, 619b, respectively. FIGS. 6A and 6B show the basket 600 coupled to the shaft 104 while FIG. 6C shows the basket 600 isolated from the shaft 104. The basket 600 comprises a mesh body 602 defining a cavity therein and a proximal edge that defines an opening or mouth 604 in communication with the cavity. The mesh body 602 can have a closed distal region 603. The basket 600 has a collapsed or constrained configuration for delivery to the treatment site through a delivery sheath, and an expanded configuration once removed from the constraints of the sheath and for capturing and removing obstructive material. The basket 600 can have collapsed and expanded diameters as detailed above with respect to basket 118.

As shown in FIGS. 6A-6E, the mesh body 602 can be formed of a plurality of interwoven and/or braided filaments (e.g., arranged in an under-over pattern). The filaments can comprise a shape-memory, resilient, and/or superelastic material (e.g., a metal, a metal alloy, nitinol, cobalt chromium alloy, a polymer, etc.) such that the mesh body 602 is self-expanding at or below body temperature.

The free ends of the filaments can be gathered and constrained at the proximal and distal hubs 619a, 619b located at the proximal and distal end portions 600a, 600b of the basket 600. The hubs 619a, 619b can be rotationally free or fixed relative to the shaft 104 and axially free or fixed relative to the shaft 104, including all of the variations disclosed above with reference to capture assembly 112. As best shown in FIG. 6B, the basket 600 may be arranged on the shaft 104 such that the proximal hub 619a is offset from a centerline along a height dimension h of the basket 600 (e.g., the shaft 104 is eccentrically positioned within the basket lumen). For example, the shaft 104 may extend through the interior cavity of the basket 600 proximate the sidewall of the basket 600, along a bottom third or bottom fourth of the basket 600. Such an offset configuration provides a substantially unobstructed opening at the mouth 604. As used herein with respect to any embodiment, a “top” side of the basket refers to the side of the basket opposite that of the shaft 104 when the capture assembly is in a relaxed state (under no external constraints). Likewise, a “bottom” side of the basket refers to a side of the basket adjacent to the shaft 104 when the capture assembly is in a relaxed state.

In some embodiments, the mesh body 602 can comprise a proximal portion 606 and a distal portion 608, each having distinct properties and functions. For example, the proximal portion 606 can be configured to provide vessel wall apposition and sealing, while the more flexible distal portion 608 can be configured to collect and retain dislodged material in the bloodstream. In some embodiments, the proximal and distal portions 606, 608 comprise different portions of a single, continuous braid (e.g., the proximal and distal portions 606, 608 are formed of the same filaments), and their distinct properties are created by varying cross-sectional shapes, filament size, filament count, pore size, pitch, pic, etc. In other embodiments, including those depicted herein, the proximal and distal portions 606, 608 are discrete structures coupled to one another at a circumferential joint 610. In such embodiments, the proximal portion 606 comprises a first or proximal plurality of interwoven and/or braided wires and the distal portion 608 comprises a second or distal plurality of interwoven and/or braided wires. Details regarding the joint 610 are discussed below with reference to FIG. 6D. In some cases, the proximal portion 606 may include a braid comprising a plurality of interwoven filaments while the distal portion 608 includes a weave comprising separate warp and weft elements. The filaments of the proximal and distal portions 606, 608 may comprise the same or different materials.

The proximal portion 606 comprises the proximalmost portion of the basket 600 and includes a proximal edge defining a mouth 604 and a sidewall 612 extending distally from the mouth 604. The ends of each of the proximal filaments are secured at the proximal hub 619a, and an intermediate portion of each of the filaments is inverted at the distal end of the proximal portion 606 (as shown in FIG. 6D). A majority (e.g., not all) of the proximal edge of the mouth 604 is defined by first and second arms 614a, 614b (referred to collectively as “arms 614”) formed of bundled portions of the filaments. As used herein, “bundled” refers to any twisting, wrapping, crimping together, or other arrangement of the filaments in which the filaments are positioned in close contact with one another. The first and second arms 614a, 614b extend distally from the hub 619a, diverging then converging before terminating at respective first and second distal ends 616a, 616b (referred to collectively as “distal ends 614”). The bundled-filament arms 614 provide increased radial strength around the mouth 604 for vessel wall apposition and sealing, without requiring a separate reinforcing structure. The bundling of the filaments along the arms 614 may also enable the use of proximal filaments all having the same length (i.e., when arranged end-to-end, prior to braiding), as the extra slack of some of the filaments is distributed along the bundle.

An enlarged, isolated view of the distal portion of the proximal edge and mouth 604 are shown in FIG. 6E. As each arm 614 extends distally, the filaments branch away from the arms 614 to form the sidewall 612 of the proximal portion 606. The distal ends 616a, 616b of the arms 614 may be substantially longitudinally aligned but are separated circumferentially from one another by a gap 618 that defines a break in the rim 614. As such, the perimeter of the mouth 604 comprises a reinforced, more rigid portion coinciding with the arms 614a, 614b and/or rim 614 and a more flexible portion coinciding with the gap 618.

Still with reference to FIG. 6E, the portion of the proximal edge coinciding circumferentially with the gap 618 (also the distalmost aspect of the edge) comprises a single filament 640 from each of the arms 614 and their intersection 644. In some embodiments, for example as shown, the final filaments 640, 642 emerging from respective distal ends 616a, 616b of the first and second arms 614a, 614b may extend distally away from the corresponding distal ends 616a, 616b at an angle with respect to the longitudinal axis great enough such that the emerging filaments 640, 642 do not extend directly across the gap 618. In some cases, at least one 642 of the emerging filaments from each of the arms 614 does not continue in the same circumferential direction as the arm 614a, 614b from which it emerged but rather bends in the opposite circumferential direction as it extends helically and wraps around the sidewall 612 while interweaving with other filaments.

In some examples, the gap 618 is disposed at a location diametrically opposed to the proximal hub 619a (e.g., at substantially the same circumferential position as the proximal end portion 618a of the basket 600), as shown. In other embodiments, the gap 618 may be at other circumferential locations along the mouth 604.

As best shown in FIG. 6B, in some examples the proximal portion 606 of the basket 600 has a first region 646 and a second region 648 having different properties, such as different PIC (“per inch crossing”) counts, braid angles, pore sizes, and others. The first region 646 may extend from a proximal end portion at the proximal hub 619a to the distalmost aspect of the proximal edge of the basket 600. The second region 648 may extend distally from a proximal end portion of the first region 646 to the proximal end portion of the distal portion 608. As such, the second region 648 comprises a circumferentially continuous portion of the sidewall 612, with the exception of the openings between the interwoven filaments. In some examples, the second region 648 has a substantially constant diameter along its length, while a height h of the first region 646 increases in a distal direction. The first region may have a first average pore size while the second region has a second average pore size less than the first average pore size. Additionally or alternatively, the first region can have a first PIC count while the second region has a second PIC count greater than the first PIC count. Additionally or alternatively, the first region can have a first longitudinal stiffness while the second region has a second longitudinal stiffness less than the first longitudinal stiffness.

As previously mentioned, in some embodiments the proximal portion 606 is formed of filaments having the same length, which enables the basket 600 to elongate evenly when radially collapsing for sheathing without portions of the basket 600 binding or bunching. The uniform wire lengths enable the basket 600 to elongate and collapse evenly despite a length along the top side 623 of the basket 600 (e.g., opposite the shaft 104) being shorter than the bottom side 625 (e.g., adjacent the shaft 104). When filaments of different lengths are used, the basket 600 may have a tendency to collapse non-uniformly when resheathed such that the extra length at the bottom side 625 of the basket 600 bunches and/or bulges, making it difficult to resheath the entire basket 600.

In some examples, the proximal portion 606 of the basket 600 can have substantially uniform properties (e.g., PIC count, braid angle, pore size, and/or others) along its length.

In some embodiments, the proximal portion 606 comprises filaments of at least two different lengths.

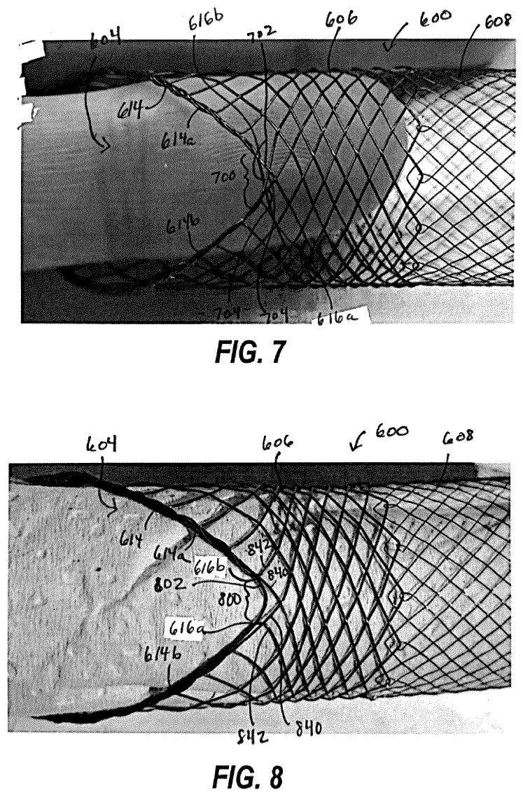

According to some embodiments, the proximal edge of the basket 600 is formed entirely of the filaments forming the sidewall 612 of the proximal portion 606 and without an additional reinforcing structure. In other embodiments, a separate reinforcing structure may be incorporated into the bundled filaments to provide additional radial strength. In such cases, the reinforcing element may span the gap 618. These and other proximal edge configurations are discussed in greater detail below with reference to FIGS. 7, 8, 9, 10A and 10B.

Still with reference to the side view of FIG. 6B, the proximal edge may be disposed at a non-90-degree angle (referred to as the “mouth angle M”) relative to the longitudinal axis L of the basket 600. In some cases, it may be beneficial to have a mouth angle M of at least 30 degrees, at least 45 degrees, or approximately 45 to 60 degrees. A mouth angle M that is too steep (e.g., greater than 45 degrees, greater than 60 degrees, etc.) makes the basket difficult to draw into the sheath, and a mouth angle M too shallow (e.g., less than 20 degrees, less than 30 degrees, etc.) increases the potential for tissue to bypass due to lighter pressure with the vessel wall.

It will be appreciated that the proximal edge configurations of the present technology can be used with any mesh basket, and not just those with discrete proximal and distal structures as shown in the drawings. For example, the present technology includes a mesh basket with a single braid construction and/or substantially uniform properties along its length, and further includes any of the proximal edge configurations disclosed herein.

The sidewall 612 of the proximal portion 606 can be stiffer and/or provide more radially outward force than that of the distal portion 608, again to help maintain apposition of the proximal portion of the basket 600 against the vessel wall. Various parameters may be adjusted to achieve such rigidity. For example, the proximal filaments may have a larger diameter, on average, than the distal filaments. In some embodiments, the proximal filaments can have a diameter of no less than 0.004 inches, no less than 0.005 inches, no less than 0.006 inches, no less than 0.007 inches, no less than 0.008 inches, about 0.007 inches, or about 0.0075 inches. The filaments can have a round, square, rectangular, or other cross-sectional shape. Additionally or alternatively, the pore size along the proximal portion 606 can be greater than that of the distal portion. For example, the proximal portion 606 can have an average pore size of from about 0.25 mm² to about 36 mm², about 1 mm² to about 36 mm², about 1 mm² to about 2 mm². at least 1 mm², at least 2 mm², at least 3 mm², at least 4 mm², at least 5 mm², at least 6 mm², at least 7 mm², at least 8 mm², at least 9 mm², at least 10 mm², and others. Additionally or alternatively, the proximal portion 606 can have a filament count that is less than a filament count of the distal portion 608. For example, the proximal portion can have a filament count of 8 to 144 filaments, 16 to 64 filaments, 16 to 44 filaments, 28 filaments, or others.

As depicted, the proximal portion 608 may be formed exclusively of interwoven and/or braided filaments (e.g., a non-unitary structure) and does not include a laser-cut tube or other monolithic structure.

The distal portion 608 of the mesh basket 600 comprises the distalmost portion of the basket 600 and includes a sidewall 620 extending distally away from the joint 610 and/or the distal end of the proximal portion 608. The ends of each of the distal filaments are secured at the distal hub 619b, and an intermediate portion of each of the filaments is inverted at the proximal end of the distal portion 608 (as shown in FIG. 6D). The distal end of the distal sidewall 620 tapers down to and is secured by the distal hub 619b. The distal portion 608 of the mesh basket 600 can be more flexible and/or provide less chronic outward force than the proximal portion 606. Unlike the proximal portion 608, the distal portion 608 is generally not relied on for anchoring and/or maintaining apposition with the blood vessel wall, and rather is configured to collect and retain dislodged emboli in the blood stream. One or more parameters of the distal portion 608 can be varied to achieve such flexibility. For example, the distal portion 608 can have a smaller pore size than the proximal structure and/or the distal filaments can have a smaller diameter than the proximal filaments. In some embodiments, the distal portion 608 has a pore size of approximately 0.25 mm² to about 36 mm², about 1 mm² to about 36 mm², about 1 mm² to about 2 mm². at least 1 mm², at least 2 mm², at least 3 mm², at least 4 mm², at least 5 mm², at least 6 mm², at least 7 mm², at least 8 mm², at least 9 mm², at least 10 mm², and others. Additionally or alternatively, the distal filaments can have an average diameter of from about 0.0020 inches to about 0.0050 inches, no more than 0.005 inches, or about 0.0035 inches. Additionally or alternatively, the distal portion 608 can have a filament count that is greater than a filament count of the proximal portion 606. For example, the distal portion 608 can have a filament count of from about 8 to 144 filaments, 16 to 64 filaments, 28 to 64 filaments, 56 filaments, or others.

In some examples, the distal portion 608 can have a first region 620 comprising a substantially constant diameter and a second region 622, distal of and extending from the first region 622, comprising a tapering diameter. In some embodiments the distal portion 608 can taper along its entire length. Tapering of the distal portion 608 may improve resheathing of the mesh basket 600 and enclosed liberated material.

FIG. 6D is an enlarged view of the joint 610 between the proximal and distal portions 606, 608. As shown, the distal end of the proximal portion 606 can be interwoven with the proximal end of the distal portion 608 to couple the proximal and distal portions 606, 608 to one another. In some examples, the proximal end of the distal portion 608 can include a plurality of loops 630 and a plurality of proximally pointing apices 632. Each of the loops 630 can comprise an inverted portion of one of the distal filaments that has been twisted over itself, and each of the apices 632 can comprise an inverted portion of a distal filament (with no additional twist). In some embodiments, each of the loops 630 is substantially circumferentially aligned with one of the apices 632, with the loop 630 positioned distally of the corresponding apex 632. The distal end of the proximal portion 606 can comprise a plurality of distally pointing apices 634, each comprising an inverted portion of one of the proximal filaments. The apices 634 of the proximal portion 606 can be threaded through the loops 630 of the distal portion 608, thereby interlocking the braid of the proximal portion 606 to the braid of the distal portion 608. Proximal movement of the proximal portion 606 away from the distal portion 608 is limited by the distal ends of the loops 630, and distal movement of the proximal portion 606 relative to the distal portion 608 is limited by the crossover joints in the loops 630. The apices 632 of the distal portion 606 may extend distally beyond the apices 634 of the proximal portion 606 and be disposed radially inwardly of the distal end of the proximal portion 608 (e.g., the apices 632 are not interwoven with the proximal filaments). In some examples, the apices 632 are also interwoven with the proximal filaments such that one leg adjacent the apex is disposed over (e.g., radially outward of) a proximal filament and the other leg adjacent the apex is disposed under (e.g., radially inward of) a proximal filament.

In some embodiments the proximal end of the distal portion 608 includes only loops 630 or only apices 632. In the latter scenario, the apices 632 would be interwoven with the proximal filaments. Other means of coupling the proximal and distal portions 606, 608 of the basket 600 are possible, such as joining by welding, encapsulation of the ends in a polymer or other material, etc.

FIG. 7 is an enlarged view of a proximal portion of a mesh basket 600, shown with an alternative mouth arrangement. As shown, in some embodiments both filaments emerging from the distal end 616a, 616b of each arm 614a, 614b continue to wrap around the circumference of the basket 600 in the same direction as the arm 614a, 614b from which it emerged, and at a lower angle such that the distalmost aspect of the proximal edge is formed of a higher density of filaments (as compared to the rest of the sidewall 612 and not including the arms 614). The distalmost aspect 700 of the proximal edge thus comprises at least two filaments 702 emerging from the first arm 614a, extending in a first circumferential direction, interwoven with at least two filaments 704 emerging from the second arm 614b, extending in a second circumferential direction opposite the first circumferential direction. The distalmost aspect 700 is thus more flexible than the arms 614, thereby providing varying rigidity along the proximal edge, while being more reinforced than the example shown in FIG. 6E. While the embodiment shown in FIG. 7 does not include a reinforcing element, in some examples this configuration may additionally include a reinforcing element.

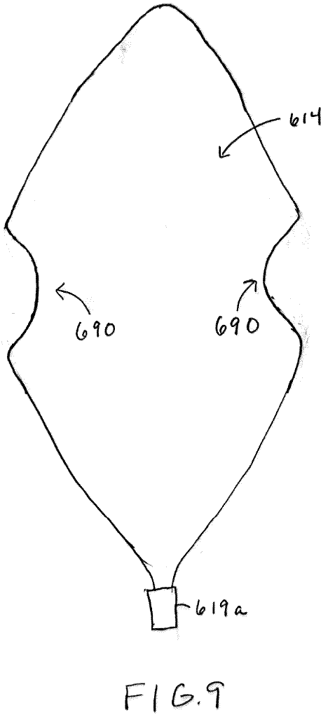

In some examples, the mesh basket may include one or more reinforcing elements disposed around all or a portion of the proximal edge. The reinforcing element can be configured to increase the radial strength around the proximal edge, thereby improving expansion of the mouth and sealing with the vessel wall, as well as improved unsheathing and resheathing. FIG. 8, for example, is an enlarged view of a proximal portion of a mesh basket 600 including a reinforcing element 802 comprising a single filament extending around the mouth 604, including along the arms 614a, 614b as well as the distalmost aspect 800 of the mouth 604 (e.g., between the distal ends 616a, 616b of the arms 614a, 614b). The reinforcing element 802 can have first and second ends at the proximal hub 619a and an intermediate portion that extends along the arms 614a, 614b and includes an inverted portion at the distalmost aspect 800 of the mouth 604. Along the arms 614a, 614b, the reinforcing element 802 may be bundled with the filaments.

In some embodiments, the reinforcing element 802 may comprise multiple (e.g., two or more) discontinuous filaments positioned around the proximal edge of the basket.

The reinforcing element 802 can be used with any mouth and/or proximal edge configuration, including those shown in FIGS. 6E, 7, 8, 9, and 10A and 10B. In FIG. 8, the reinforcing element 802 is shown incorporated into a proximal edge similar to the mouth of FIG. 6E. As shown, at least one 642 of the emerging filaments from each of the arms 614a, 614b does not continue in the same circumferential direction as the arm 614a, 614b from which it emerges but rather is bent and redirected such that it winds distally around the basket 600 (interwoven with the other proximal filaments) in a circumferential direction opposite of the arm 614a, 614b from which it emerged. The other emerging filament 840 may continue in the same direction as the arm 614a, 614b from which it emerged.

According to some examples, the reinforcing element 802 comprises a single filament having a diameter of from about 0.1 mm to about 0.3 mm, or about 0.2 mm, or about 0.22 mm. In some embodiments, the reinforcing element 802 may comprise a plurality of filaments (e.g., bunched, twisted, braided, etc.) or a structure cut from a metal sheet or tube. The reinforcing element 802 can comprise a shape-memory, resilient, and/or superelastic material (e.g., a metal, a metal alloy, nitinol, cobalt chromium alloy, a polymer, etc.) such that it is self-expanding at or below body temperature and/or can be shape set.

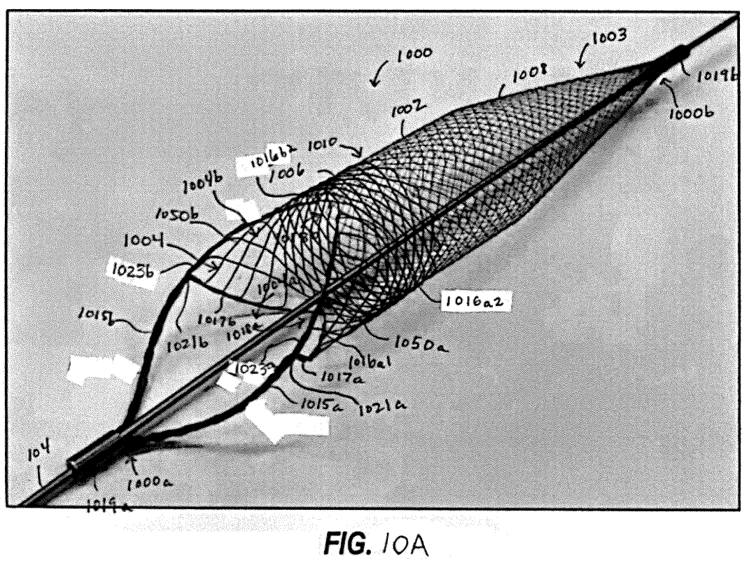

In some examples, the reinforcing element 802 is shape set into a desired mouth shape. The shape could be that shown in the drawings (e.g., ovular, diamond-shaped, etc.), or others. In some cases, one or more portions of the reinforcing element 802 flares inwardly or outwardly (e.g., the concavity of the reinforcing element and/or proximal edge varies). For example, FIG. 9 shows a proximal edge having tabs 690 that extend radially inwardly from the immediately adjacent lengths of the proximal edge. Such tabs 690 may beneficially help retain captured material within the interior cavity of the basket during withdrawal of the basket 600 from the body. Each of the tabs 690 can be convex towards the mouth 614. The tabs 690 may be formed by shape-setting the bundled filaments and/or by inclusion of a reinforcing element(s) 802 having a preset shape that includes the tabs 690.

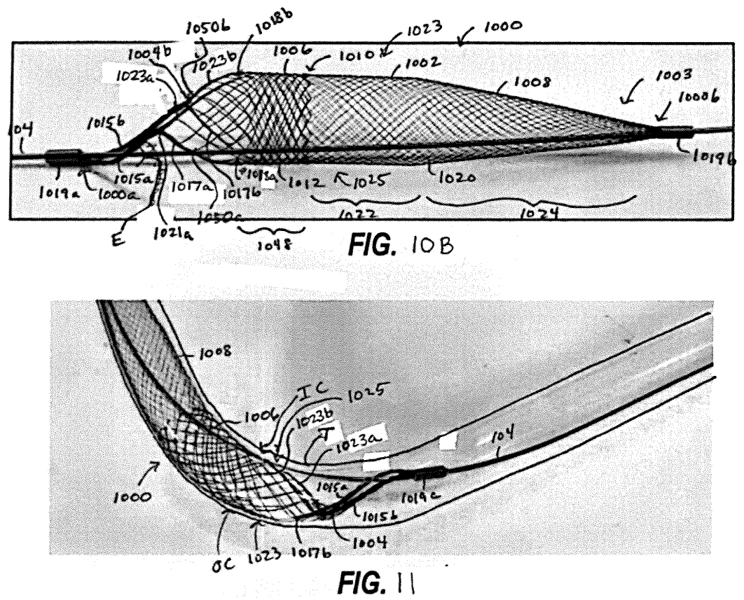

FIGS. 10A and 10B show perspective and side views, respectively, of another mesh basket 1000 (or “basket 1000”) configured in accordance with the present technology. The mesh basket 1000 has proximal and distal end portions 1000a, 1000b configured to be coupled to the shaft 104 of the capture device 107 (FIGS. 1A and 1B) at proximal and distal hubs 1019a, 1019b, respectively. The basket 1000 comprises a mesh body 1002 defining a cavity therein and having a closed distal region 1003 and an opening or mouth 1004 at the proximal region defined by a proximal edge of the basket 1000. The basket 1000 has a collapsed or constrained configuration for delivery to the treatment site through a sheath, and an expanded configuration once removed from the constraints of the sheath and for capturing and removing obstructive material. The basket 1000 can have collapsed and expanded diameters as detailed above with respect to basket 118.

As shown in FIGS. 10A and 10B, the mesh body 1002 can be formed of a plurality of interwoven and/or braided filaments (e.g., arranged in an under-over pattern). The filaments can comprise a shape-memory, resilient, and/or superelastic material (e.g., a metal, a metal alloy, nitinol, cobalt chromium alloy, a polymer, etc.) such that the mesh body 1002 is self-expanding at or below body temperature. The free ends of the filaments can be gathered and constrained at the hubs 1019a, 1019b located at the proximal and distal end portions 1000a, 1000b of the basket 1000. The hubs 1019a, 1019b can be rotationally free or fixed relative to the shaft 104 and axially free or fixed relative to the shaft 104, including all of the variations disclosed above with reference to capture assembly 112. As best shown in FIG. 10B, the basket 1000 may be arranged on the shaft 104 such that the proximal hub 1019a is offset from a centerline along a height dimension h (see FIG. 6B) of the basket 1000 (e.g., the shaft 104 is eccentrically positioned within the basket lumen). For example, the shaft 104 may extend through the interior cavity of the basket 1000 proximate the sidewall of the basket 1000, along a bottom third or bottom fourth of the basket 1000. Such an offset configuration provides a substantially unobstructed opening at the mouth 1004.

In some embodiments, the mesh body 1002 can comprise a proximal portion 1006 and a distal portion 1008, each having distinct properties and functions. For example, the proximal portion 1006 can be configured to provide vessel wall apposition and sealing, while the more flexible distal portion 1008 can be configured to collect and retain dislodged material in the bloodstream. In some embodiments, the proximal and distal portions 1006, 1008 comprise different portions of a single, continuous braid (e.g., the proximal and distal portions 1006, 1008 are formed of the same filaments), and their distinct properties are created by varying cross-sectional shapes, pore size, pitch, etc. In other embodiments, including those depicted herein, the proximal and distal portions 1006, 1008 are discrete structures coupled to one another at a circumferential joint 1010. In such embodiments, the proximal portion 1006 comprises a first or proximal plurality of interwoven and/or braided wires and the distal portion 1008 comprises a second or distal plurality of interwoven and/or braided wires. Details regarding the joint 1010 are discussed above with reference to FIG. 6D. In some cases, the proximal portion 1006 may include a braid comprising a plurality of interwoven filaments while the distal portion 1008 includes a weave comprising separate warp and weft elements. The filaments of the proximal and distal portions 1006, 1008 may comprise the same or different materials.

The proximal portion 1006 comprises the proximalmost portion of the mesh basket 1000 and includes the proximal edge, the mouth 1004, and a sidewall 1012 extending distally from the proximal edge. The ends of each of the proximal filaments are secured at the proximal hub 1019a, and an intermediate portion of each of the filaments is inverted at the distal end of the proximal portion 1006 (as shown in FIG. 6D).

The proximal portion of the basket 1000 also includes first and second legs 1015a, 105b formed exclusively of bundled proximal filaments. Unlike the arms 1014a, 1014b of basket 600, the filaments do not split off and/or diverge from the bundle defining each leg 1015a, 1015b. Each leg 1015a, 1015b has a proximal end at the proximal hub 1019a and a distal end at a corresponding branch point 1021a, 1021b, at which the bundled filaments branch into first and second arms 1017, 1023 (labeled individually as 1017a, 1017b, 1023a, and 1023b). The first arms 1017a, 1017b break towards a lower surface of the basket 1000, along which the shaft 104 extends. The second arms 1023a, 1023b break towards an upper surface of the basket 1000. Along each of the first and second arms 1017, 1023, the filaments split away from the bundle to form the sidewall 1012 of the proximal portion 1006. The filaments of the first and second arms 1017a, 1023a define a first prong 1050a, and the filaments of the first and second arms 1017b, 1023b define a second prong 1050b.

As best shown in FIG. 10B, in some examples the proximal portion 1006 of the basket 1000 has a first region 1046 and a second region 1048 having different properties, such as different PIC counts, braid angles, pore sizes, and others. The first region 1046 may include the legs 1015a, 1015b and the prongs 1050a, 1050b, and the second region 1048 may comprise the portion of the sidewall 1012 between the distal end of the prongs 1050a, 1050b and the proximal end portion of the second region 1048. The second region 1048 may comprise a circumferentially continuous portion of the sidewall 1012, with the exception of the openings between the interwoven filaments. In some examples, the second region 1048 has a substantially constant diameter along its length. The prongs 1050a, 1050b may have a first average pore size while the second region 1048 has a second average pore size less than the first average pore size. Additionally or alternatively, the prongs 1050a, 1050b can have a first PIC count while the second region 1048 has a second PIC count greater than the first PIC count. Additionally or alternatively, the prongs 1050a, 1050b can have a first longitudinal stiffness while the second region 1048 has a second longitudinal stiffness less than the first longitudinal stiffness.

The first arms 1017a, 1017b can extend distally from corresponding first and second branch points 1021a, 1021b to corresponding distal ends 1016a1, 1016b1 (only 1016a1 labeled in FIG. 10A) that are substantially longitudinally aligned but separated circumferentially from one another by a first gap 1018a. A first mouth 1004a is thus defined by the first arms 1017a, 1017b and the filaments at the distal aspect between them. As such, the portion of the proximal edge surrounding the first mouth 1004a comprises a reinforced, more rigid portion coinciding with the first arms 1017a, 1017b and a more flexible portion coinciding with the first gap 1018a. The filaments emerging from the distal ends 1016a1, 1016b1 of each of the first arms 1017a, 1017b may extend distally away from the corresponding distal ends 1016a1, 1016b1 at enough of an angle such that the emerging filaments do not extend directly across the first gap 1018a. In some cases, at least one of the emerging filaments from each of the first arms 1017a, 1017b does not continue in the same circumferential direction as the arm 1017a, 1017b from which it emerged but rather is bent and redirected such that it winds distally around the basket 1000 (interwoven with the other proximal filaments) in an opposite circumferential direction as the arm 1017a, 1017b from which it emerged. As a result, the portion of the sidewall 1012 defining the distalmost aspect of the first mouth 1004a comprises a single filament extending from each arm 1017a, 1017b, and the crossover point between the two.

The bundled-filament first arms 1017a, 1017b provide increased radial strength around the first mouth 1004a, thereby improving vessel wall apposition and sealing and reducing or eliminating the risk of gaps between the rim and the wall. In some examples, the first gap 1018a is disposed at substantially the same height and/or circumferential location as the proximal hub 1019a, as shown. In other embodiments, the first gap 1018a may be positioned at other locations relative to the proximal hub 1019a. For example, the first and second prongs 1050a, 1050b, and thus the first and second gaps 1018a, 1018b, may be rotated 90 degrees.

The second arms 1023a, 1023b extend distally from corresponding first and second branch points 1021a, 1021b to corresponding distal ends 1016a2, 1016b2 (labeled in FIG. 10A only) that are substantially longitudinally aligned but are separated circumferentially from one another by a second gap 1018b. A second mouth 1004b is thus defined by the second arms 1023a, 1023b and the filaments at the distal aspect between them. In some embodiments, the first and second mouths 1004a, 1004b may be diametrically opposed from one another. As such, the portion of the proximal edge around the second mouth 1004b comprises a reinforced, more rigid portion coinciding with the second arms 1023a, 1023b and/or rim and a more flexible portion coinciding with the second gap 1018b. The filaments emerging from the distal ends 1016a2, 1016b2 of each of the second arms 1023a, 1023b may extend distally away from the corresponding distal ends 1016a2, 1016b2 at enough of an angle such that the emerging filaments do not extend directly across the second gap 1018b. In some cases, at least one of the emerging filaments from each of the second arms 1023a, 1023b does not continue in the same circumferential direction as the arm 1023a, 1023b from which it emerged but rather is bent and redirected such that it winds distally around the basket 1000 (interwoven with the other proximal filaments) in an opposite circumferential direction as the arm 1023a, 1023b from which it emerged. As a result, in some embodiments the portion of the sidewall 1012 defining the distalmost aspect of the second mouth 1004b comprises a single filament extending from each arm 1023a, 1023b, and the crossover point between the two.

The bundled-filament second arms 1023a, 1023b provide increased radial strength around the second mouth 1004b, thereby improving vessel wall apposition and sealing and reducing or eliminating the risk of gaps between the rim and the wall. In some examples, the second gap 1018b is at a circumferential location that is diametrically opposite the proximal hub 1019a, as shown. In other embodiments, the second gap 1018b may be positioned at other locations relative to the proximal hub 1019a. For example, the first and second prongs 1050a, 1050b, and thus the first and second gaps 1018a, 1018b, may be rotated 90 degrees.

The proximal edge defining the first mouth 1004a may be formed entirely of the filaments forming the sidewall of the proximal portion 1006 and does not include an additional reinforcing structure. Additionally or alternatively, the proximal edge of the second mouth 1004b may be formed entirely of the filaments forming the sidewall of the proximal portion 1006 and does not include an additional reinforcing structure. In some embodiments, a separate reinforcing structure may be incorporated into the bundled filaments of the first and/or second proximal edges to provide additional radial strength. These and other mouth configurations are discussed in greater detail above with reference to FIGS. 7, 8, 9, 10A, and 10B.

With reference to the side view of FIG. 10B, the legs 1015a, 1015b may be disposed at a non-90-degree angle (referred to as the “leg angle E”) relative to the longitudinal axis L of the basket 1000. In some cases, it may be beneficial to have an leg angle E of at least 30 degrees, at least 45 degrees, or approximately 45 to 60 degrees when treating vessel sizes greater than 25 mm. A leg angle E that is too steep (e.g., greater than 45 degrees, greater than 60 degrees, etc.) makes the basket difficult to draw into the sheath, and a mouth angle M too shallow (e.g., less than 20 degrees, less than 30 degrees, etc.) increases the potential for tissue to bypass due to lighter pressure with the vessel wall.

The sidewall 1012 of the proximal portion 1006 can be stiffer and/or provide more radially outward force than that of the distal portion 1008, again to help maintain apposition of the proximal portion of the basket 1000 against the vessel wall. Various parameters may be adjusted to achieve such rigidity. For example, the proximal filaments may have a larger diameter, on average, than the distal filaments. In some embodiments, the proximal filaments can have a diameter of no less than 0.004 inches, no less than 0.005 inches, no less than 0.006 inches, no less than 0.007 inches, no less than 0.008 inches, about 0.007 inches, or about 0.0075 inches. The filaments can have a round, square, rectangular, or other cross-sectional shape. Additionally or alternatively, the pore size along the proximal portion 1006 can be greater than that of the distal portion. For example, the proximal portion 1006 can have a pore size of from about 0.25 mm² to about 36 mm², about 1 mm² to about 36 mm², about 1 mm² to about 2 mm². at least 1 mm², at least 2 mm², at least 3 mm², at least 4 mm², at least 5 mm², at least 6 mm², at least 7 mm², at least 8 mm², at least 9 mm², at least 10 mm², and others. Additionally or alternatively, the proximal portion 1006 can have a filament count that is less than a filament count of the distal portion 1008. For example, the proximal portion can have a filament count of from about 8 to 144 filaments, 16 to 64 filaments, 16 to 44 filaments, 28 filaments, or others. The filaments of the proximal portion 1006 can have the same length (i.e., when laid straight) such that the structures elongate evenly when collapsed for low-profile sheathing without filament binding or bunching, as detailed herein. In other embodiments, at least some of the proximal filaments can have different lengths.

The prongs 1050 of the present technology can be formed exclusively of interwoven and/or braided filaments (e.g., a non-unitary structure) and do not include a laser-cut tube or other monolithic structure.

The distal portion 1008 of the mesh basket 1000 can be generally similar to the distal portion 608 discussed above with reference to FIGS. 6A-6E and is labeled with similar numerals. For example, sidewall 620 is labeled as sidewall 1020 in FIGS. 10A and 10B. Likewise, the joint 1010 of the mesh basket 1000 can be generally similar to the joint 610 discussed above with reference to FIGS. 6A-6E, including all variations disclosed herein.

FIG. 11 shows the mesh basket 1000 positioned around a curve in a tube T. As shown, the first and second legs 1015a, 1015b space the proximal edge of the basket 1000 longitudinally from the connection to the shaft 104, thereby mechanically decoupling the proximal edge from the shaft 104 such that, when the basket 1000 is positioned around a curve, the proximal edge remains in firm contact with an inner surface of the blood vessel. As such, the first and second legs 1015a, 1015b are configured to position the basket 1000 within the blood vessel lumen independent of the path of the shaft 104. In some embodiments, the proximal edge of the basket 1000 maintains contact with the vessel wall when the basket 1000 is positioned around a curve having a radius of curvature as small as 20 mm, and in some cases as small as 10 mm.

In FIG. 11, the mesh basket 1000 is positioned around a curve such that the bottom side 1025 of the basket 1000 and the shaft 104 are adjacent the inner aspect IC of the curve while the top side 1023 is adjacent the outer aspect OC of the curve. When the capture assembly is positioned around a curve having the opposite curvature, the legs 1015a, 1015b allow the basket 1000 to pivot relative to the shaft 104 such that the top side 1023 of the basket 1000 and the shaft 104 are adjacent the inner aspect IC of the curve and the bottom side 1025 is adjacent the outer aspect OC of the curve, still with the proximal edge in contact with the inner surface of the blood vessel.

### CONCLUSION

Although many of the embodiments are described above with respect to systems, devices, and methods for retrieving clot material from a blood vessel lumen, the technology is applicable to other applications and/or other approaches, such as removal and/or modification of other obstructive structures within any body lumen (e.g., crossing catheters, thrombectomy devices, atherectomy devices, etc.). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-11.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for capturing material in a blood vessel, the device comprising:
an elongate control member having a proximal portion and a distal portion, the distal portion configured to be intravascularly at a treatment site in a blood vessel lumen proximate an obstruction;
an expandable basket coupled to the distal portion of the control member with the control member extending longitudinally through the basket, the basket comprising a plurality of braided filaments defining an interior cavity, wherein the basket has a closed distal end portion and an opening defined by a proximal edge of the basket, the opening being in communication with the interior cavity; and
first and second legs having respective proximal ends coupled to the distal portion of the control member at a connection and distal ends at the proximal edge of the basket, wherein each of the first and second legs are formed of bundled portions of the filaments,
wherein the first and second legs are configured to position the basket within the blood vessel lumen independent of the path of the control member such that the proximal edge of the basket remains in contact with an inner surface of the blood vessel lumen when the basket is positioned around a curve.

2. The device of claim 1, wherein the bundled filaments of the first and second legs branch at distal ends of the first and second legs into respective upper and lower arms, wherein each of the upper arms and each of the lower arms are formed of bundled filaments.

3. The device of claim 2, wherein the filaments branch distally away from the filament bundle of the respective upper and lower arms to form the sidewall of the basket.

4. The device of claim 3, wherein:
the first leg splits at its distal end into a first upper arm and a first lower arm, the first upper arm and first lower arm defining a first prong, and
the second leg splits at its distal end into a second upper arm and a second lower arm, the second upper arm and second lower arm defining a second prong.

5. The device of claim 4, wherein the first and second upper arms converge along a circumferential direction towards one another as the first and second upper arms extend distally, and wherein the distal ends of the first and second upper arms are spaced apart from one another along a circumferential direction by a gap.

6. The device of claim 5, wherein the basket includes a reinforcing element extending along the first and second upper arms, with the bundled filaments, and spanning the circumferential gap between the distal ends of the first and second upper arms.

7. The device of any one of claims 4 to 6, wherein the first and second lower arms converge along a circumferential direction towards one another as the first and second lower arms extend distally, and wherein the distal ends of the first and second lower arms are spaced apart from one another along a circumferential direction by a gap.

8. The device of claim 7, wherein the basket includes a reinforcing element extending along the first and second lower arms, with the bundled filaments, and spanning the circumferential gap between the distal ends of the first and second lower arms.

9. The device of any one of claims 4 to 8, wherein the first and second prongs are diametrically opposed.

10. The device of any one of claims 4 to 9, wherein a sidewall of the basket has a first region comprising the first and second prongs and a second region extending distally from the first and second prongs, and wherein the sidewall is circumferentially continuous along the second region, with the exception of the openings between the interwoven filaments.

11. The device of claim 10, wherein the first and second prongs have a first average pore size and the second region has a second average pore size less than the first average pore size.

12. The device of claim 10 or claim 11, wherein the first and second prongs have a first pic count and the second region has a second pic count greater than the first pic count.

13. The device of any one of claims 1 to 12, wherein the filaments do not diverge away from the bundle along the legs.

14. The device of any one of claims 1 to 13, wherein the basket comprises a proximal structure and a distal structure coupled to the proximal structure at a circumferential joint.

15. The device of claim 14, wherein:
the filaments are first filaments and the proximal structure is formed of the first filaments, and
the distal structure is formed of a plurality of interwoven second filaments.
